(19) United States
(12) Patent Application Publication
(10) Pub. No.: US 2009/0162356 A1
(43) Pub. Date: Jun. 25, 2009

(54) CRIg antagonists

(76) Inventor: Menno Van Lookeren Campagne, San Francisco, CA (US)

Correspondence Address: Goodwin Procter LLP;Attn: Patent Administrator, 135 Commonwealth Drive, Menlo Park, CA, 94025-1105, US

(21) Appl. No.: 12/150,220

(22) Filed: Apr. 25, 2008

Related U.S. Application Data
(60) Provisional application No. 60/915,340, filed on May 1, 2007.

(51) Int. Cl. A61K 39/395; C07K 16/28; C12N 15/13; A61P 31/18; A61P 31/14; A61P 31/20; A61P 31/22; A61P 31/04; A61P 31/10; A61P 33/00

(52) U.S. Cl. 424/136.1; 530/387.9; 530/387.3; 424/133.1; 424/139.1; 536/23.53

(57) ABSTRACT

The present invention concerns blocking antibodies specifically binding a recently discovered macrophage specific receptor, CRIg, and their use to prevent cellular entry of intracellular pathogens, such as viruses, bacteria or parasites as well as for prevention of unwanted clearance of erythrocytes and platelets.

### CROSS REFERENCE TO RELATED APPLICATIONS

This application is a non-provisional application filed under 37 CFR 1.53(b)(1), claiming priority under 35 USC 119(e) to provisional application No. 60/915,340 filed May 1, 2007, the contents of which are incorporated herein by reference.

### FIELD OF THE INVENTION

The present invention concerns blocking antibodies specifically binding a recently discovered macrophage specific receptor, CRIg, and their use to prevent cellular entry of intracellular pathogens, such as viruses, bacteria or parasites as well as for prevention of unwanted clearance of erythrocytes and platelets.

### BACKGROUND OF THE INVENTION

The Complement System

The complement system is a complex enzyme cascade made up of a series of serum glycoproteins, that normally exist in inactive, pro-enzyme form. Two main pathways, the classical and the alternative pathway, can activate complement, which merge at the level of C3, where two similar C3 convertases cleave C3 into C3a and C3b.

Macrophages are specialist cells that have developed an innate capacity to recognize subtle differences in the structure of cell-surface expressed identification tags, so called molecular patterns (Taylor, et al., Eur J Immunol 33, 2090-1097 (2003); Taylor, et al., Annu Rev Immunol 23, 901-944 (2005)). While the direct recognition of these surface structures is a fundamental aspect of innate immunity, opsonization allows generic macrophage receptors to mediate engulfment, increasing the efficiency and diversifying recognition repertoire of the phagocyte (Stuart and Ezekowitz, Immunity 22, 539-550 (2005)). The process of phagocytosis involves multiple ligand-receptor interactions, and it is now clear that various opsonins, including immunoglobulins, collectins, and complement components, guide the cellular activities required for pathogen internalization through interaction with macrophage cell surface receptors (reviewed by Aderem and Underhill, Annu Rev Immunol 17, 593-623 (1999); Underhill and Ozinsky, Annu Rev Immunol 20, 825-852 (2002)). While natural immunoglobulins encoded by germline genes can recognize a wide variety of pathogens, the majority of opsonizing IgG is generated through adaptive immunity, and therefore efficient clearance through Fc receptors is not immediate (Carroll, Nat Immunol 5, 981-986 (2004)). Complement, on the other hand, rapidly recognizes pathogen surface molecules and primes the particle for uptake by complement receptors (Brown, Infect Agents Dis 1, 63-70 (1991)).

Complement consists of over 30 serum proteins that opsonize a wide variety of pathogens for recognition by complement receptors. Depending on the initial trigger of the cascade, three pathways can be distinguished (reviewed by (Walport, N Engl J Med 344, 1058-1066 (2001)). All three share the common step of activating the central component C3, but they differ according to the nature of recognition and the initial biochemical steps leading to C3 activation. The classical pathway is activated by antibodies bound to the pathogen surface, which in turn bind the C1q complement component, setting off a serine protease cascade that ultimately cleaves C3 to its active form, C3b. The lectin pathway is activated after recognition of carbohydrate motifs by lectin proteins. To date, three members of this pathway have been identified: the mannose-binding lectins (MBL), the SIGN-R1 family of lectins and the ficolins (Pyz et al., Ann Med 38, 242-251 (2006)) Both MBL and ficolins are associated with serine proteases, which act like C1 in the classical pathway, activating components C2 and C4 leading to the central C3 step. The alternative pathway contrasts with both the classical and lectin pathways in that it is activated due to direct reaction of the internal C3 ester with recognition motifs on the pathogen surface. Initial C3 binding to an activating surface leads to rapid amplification of C3b deposition through the action of the alternative pathway proteases Factor B and Factor D. Importantly, C3b deposited by either the classical or the lectin pathway also can lead to amplification of C3b deposition through the actions of Factors B and D. In all three pathways of complement activation, the pivotal step in opsonization is conversion of the component C3 to C3b. Cleavage of C3 by enzymes of the complement cascades exposes the thioester to nucleophilic attack, allowing covalent attachment of C3b onto antigen surfaces via the thioester domain. This is the initial step in complement opsonization. Subsequent proteolysis of the bound C3b produces iC3b, C3c and C3dg, fragments that are recognized by different receptors (Ross and Medof, Adv Immunol 37, 217-267 (1985)). This cleavage abolishes the ability of C3b to further amplify C3b deposition and activate the late components of the complement cascade, including the membrane attack complex, capable of direct membrane damage. However, macrophage phagocytic receptors recognize C3b and its fragments preferentially; due to the versatility of the ester-bond formation, C3-mediated opsonization is central to pathogen recognition (Holers et al., Immunol Today 13, 231-236 (1992)), and receptors for the various C3 degradation products therefore play an important role in the host immune response.

C3 itself is a complex and flexible protein consisting of 13 distinct domains. The core of the molecule is made up of 8 so-called macroglobulin (MG) domains, which constitute the tightly packed α and β chains of C3. Inserted into this structure are CUB (C1r/C1s, Uegf and Bone mophogenetic protein-1) and TED domains, the latter containing the thioester bond that allows covalent association of C3b with pathogen surfaces. The remaining domains contain C3a or act as linkers and spacers of the core domains. Comparison of C3b and C3c structures to C3 demonstrate that the molecule undergoes major conformational rearrangements with each proteolysis, which exposes not only the TED, but additional new surfaces of the molecule that can interact with cellular receptors (Janssen and Gros, Mol Immunol 44, 3-10 (2007)).

Complement C3 Receptors on Phagocytic Cells

There are three known gene superfamilies of complement receptors: The short consensus repeat (SCR) modules that code for CR1 and CR2, the beta-2 integrin family members CR3 and CR4, and the immunoglobulin Ig-superfamily member CRIg.

CR1 is a 180-210 kDa glycoprotein consisting of 30 Short Consensus Repeats (SCRs) and plays a major role in immune complex clearance. SCRs are modular structures of about 60 amino acids, each with two pairs of disulfide bonds providing structural rigidity. High affinity binding to both C3b and C4b occurs through two distinct sites, each composed of 3 SCRs) reviewed by (Krych-Goldberg and Atkinson, Immunol Rev 180, 112-122 (2001)). The structure of the C3b binding site, contained within SCR 15-17 of CR1 (site 2), has been determined by MRI (Smith et al., Cell 108, 769-780 (2002)), revealing that the three modules are in an extended head-to-tail arrangement with flexibility at the 16-17 junction. Structure-guided mutagenesis identified a positively charged surface region on module 15 that is critical for C4b binding. This patch, together with basic side chains of module 16 exposed on the same face of CR1, is required for C3b binding. The main function of CR1, first described as an immune adherence receptor (Rothman et al., J Immunol 115, 1312-1315 (1975)), is to capture ICs on erythrocytes for transport and clearance by the liver (Taylor et al., Clin Immunol Immunopathol 82, 49-59 (1997)). There is a role in phagocytosis for CR1 on neutrophils, but not in tissue macrophages (Sengelov et al., J Immunol 153, 804-810 (1994)). In addition to its role in clearance of immune complexes, CR1 is a potent inhibitor of both classical and alternative pathway activation through its interaction with the respective convertases (Krych-Goldberg and Atkinson, 2001, supra; Krych-Goldberg et al., J Biol Chem 274, 31160-31168 (1999)). In the mouse, CR1 and CR2 are two products of the same gene formed by alternative splicing and are primarily associated with B-lymphocytes and follicular dendritic cells and function mainly in regulating B-cell responses (Molina et al., 1996). The mouse functional equivalent of CR1, Crry, inactivates the classical and alternative pathway enzymes and acts as an intrinsic regulator of complement activation rather than as a phagocytic receptor (Molina et al., Proc Natl Acad Sci USA 93, 3357-3361 (1992)).

CR2 (CD21) binds iC3b and C3dg and is the principal complement receptor that enhances B cell immunity (Carroll, Nat Immunol 5, 981-986 (2004); Weis et al., Proc Natl Acad Sci USA 81, 881-885 (1984)). Uptake of C3d-coated antigen by cognate B cells results in an enhanced signal via the B cell antigen receptor. Thus, coengagement of the CD21-CD19-CD81 coreceptor with B cell antigen receptor lowers the threshold of B cell activation and provides an important survival signal (Matsumoto et al., J Exp Med 173, 55-64 (1991)). The CR2 binding site on iC3b has been mapped partly on the interface between the TED and the MGI domains (Clemenza and Isenman, J Immunol 165, 3839-3848 (2000)).

CR3 and CR4 are transmembrane heterodimers composed of an alpha subunit (CD11b or α_{M} and CD11c or α_{X}, respectively) and a common beta chain (CD18 or β₂), and are involved in adhesion to extracellular matrix and to other cells as well as in recognition of iC3b. They belong to the integrin family and perform functions not only in phagocytosis, but also in leukocyte trafficking and migration, synapse formation and costimulation (reviewed by (Ross, Adv Immunol 37, 217-267 (2000)). Integrin adhesiveness is regulated through a process called inside-out signaling, transforming the integrins from a low- to a high-affinity binding state (Liddington and Ginsberg, J Cell Biol 158, 833-839 (2002)). In addition, ligand binding transduces signals from the extracellular domain to the cytoplasm. The binding sites of iC3b have been mapped to several domains on the alpha chain of CR3 and CR4 (Diamond et al., J Cell Biol 120, 1031-1043 (1993); Li and Zhang, J Biol Chem 278, 34395-34402 (2003); Xiong and Zhang, J Biol Chem 278, 34395-34402 (2001)). The multiple ligands for CR3: iC3b, beta-glucan and ICAM-1, seem to bind to partially overlapping sites contained within the I domain of CD11b (Balsam et al., 1998; Diamond et al., 1990; Zhang and Plow, 1996). Its specific recognition of the proteolytically inactivated form of C3b, iC3b, is predicted based on structural studies that locate the CR3 binding sites to residues that become exposed upon unfolding of the CUB domain in C3b (Nishida et al., Proc Natl Acad Sci USA 103, 19737-19742 (2006)), which occurs upon α′ chain cleavage by the complement regulatory protease, Factor 1.

CRIg is a novel macrophage associated receptor with homology to A33 antigen and JAM1. A human CRIg protein was first cloned from a human fetal cDNA library using degenerate primers recognizing conserved Ig domains of human JAM1. Sequencing of several clones revealed an open reading frame of 400 amino acids. Blast searches confirmed similarity to Z391g, a type 1 transmembrane protein (Langnaese et al., Biochim Biophys Acta 1492 (2000) 522-525). The extracellular region of this molecule was found to consist of two Ig-like domains, comprising an N-terminal V-set domain and a C-terminal C2-set domain. The novel human protein was originally designated as a “single transmembrane Ig superfamily member macrophage associated” (huSTIgMA). (huSTIgMA). Subsequently, using 3′ and 5′ primers, a splice variant of huSTIgMA was cloned, which lacks the membrane proximal IgC domain and is 50 amino acids shorter. Accordingly, the shorter splice variant of this human protein was designated huSTigMAshort. The amino acid sequence of huSTIgMA (referred to as PRO362) and the encoding polynucleotide sequence are disclosed in U.S. Pat. No. 6,410,708, issued Jun. 25, 2002. In addition, both huSTIgMA and huSTIgMAshort, along with the murine STIgMA (muSTIgMA) protein and nucleic acid sequences, are disclosed in PCT Publication WO 2004031105, published Apr. 15, 2004.

The Kupffer cells (KCs), residing within the lumen of the liver sinusoids, form the largest population of macrophages in the body. Although KCs have markers in common with other tissue resident macrophages, they perform specialized functions geared towards efficient clearance of gut-derived bacteria, microbial debris, bacterial endotoxins, immune complexes and dead cells present in portal vein blood draining from the microvascular system of the digestive tract (Bilzer et al., Liver Int 26, 1175-1186 (2006)). Efficient binding of pathogens to the KC surface is a crucial step in the first-line immune defense against pathogens (Benacerraf et al., J Exp Med 110, 27-48 (1959)). A central role for KCs in the rapid clearance of pathogens from the circulation is illustrated by the significantly increased mortality in mice depleted of KCs (Hirakata et al., Infect Immun 59, 289-294 (1991)). The identification of CRIg further stresses the critical role of complement and KCs in the first line immune defense against circulating pathogens.

The only complement C3 receptors identified on mouse KCs are CRIg and CR3 (Helmy et al., Cell 124, 915-927 (2006)), while human KCs show additional expression of CR1 and CR4 (Hinglais et al., 1989). Both CRIg and CR3 on KCs contribute to binding to iC3b opsonized particles in vitro (Helmy et al., Lab Invest 61, 509-514 (2006)). In vivo, a role of KC-expressed CR3 in the binding to iC3b-coated pathogens is less clear. CR3 has been proposed to contribute to clearance of pathogens indirectly via recruitment of neutrophils and interaction with neutrophil-expressed ICAM1 (Conlan and North, Exp Med 179, 259-268 (1994); Ebe et al., Pathol Int 49, 519-532 (1999); Gregory et al., J Immunol 157, 2514-2520 (1996); Gregory and Wing, J Leukoc Biol 72, 239-248 (2002); Rogers and Unanue, Infect Immun 61, 5090-5096 (1993)). In contrast, CRIg performs a direct role by capturing pathogens that transit through the liver sinusoidal lumen (Helmy et al., 2006, supra). A difference in the biology of CRIg vs CR3 is in part reflected by difference in binding characteristics of these two receptors. CRIg expressed on KCs constitutively binds to monomeric C3 fragments whereas CR3 only binds to iC3b-opsonized particles (Helmy et al., 2006, supra). The capacity of CRIg to efficiently capture monomeric C3b and iC3b as well as C3b/iC3b-coated particles reflects the increased avidity created by a multivalent interaction between CRIg molecules concentrated at the tip of membrane extentions of macrophages (Helmy et al., 2006, supra) and multimers of C3b and iC3b present on the pathogen surface. While CR3 only binds iC3b-coated particles, CRIg additionally bind to C3b, the first C3 cleavage product formed on serum-opsonized pathogens (Croize et al., Infect Immun 61, 5134-5139 (1993)). Since a large number of C3b molecules bound to the pathogen surface are protected from cleavage by factor H and I (Gordon et al., J Infect Dis 157, 697-704 (1988)), recognition of C3b ligands by CRIg ensures rapid binding and clearance. Thus, while both CRIg and CR3 are expressed on KCs, they show different ligand specificity, distinct binding properties and distinct kinetics of pathogen clearance.

Examples of pathogens that exploit cell surface receptors for cellular entry are viruses like human immunodeficiency virus (HIV), and intracellular bacteria like Mycobacterium tuberculosum, Mycobacterium leprae, Yersinia pseudotuberculosis, Salmonella typhimuriuni and Listeria Monocytogenes and parasites like the prostigmatoid Leishmania major (Cossart and Sansonetti, Science 304:242-248 (2004); Galan, Cell 103:363-366 (2000); Hornef et al., Nat. Immunol. 3:1033-1040 (2002); Stoiber et al., Mol. Immunol. 42:153-160 (2005)).

Although the complement system is designed to kill pathogens and to accelerate phagocytosis, it also mediates cellular entry of pathogens by as yet unknown complement receptors. CRIg is believed to serve as one of the receptors mediating complement-dependent cellular entry of intracellular pathogens.

### SUMMARY OF THE INVENTION

The present invention concerns anti-CRIg antibodies that block the binding of C3-opsonized pathogens to the cell surface, and their use to prevent cellular entry and/or block survival of such pathogens.

In one aspect the invention concerns a CRIg antagonist blocking the binding of a native sequence CRIg polypeptide to C3b and/or iC3b, and inhibiting CRIg-mediated complement-dependent cellular entry of an intracellular pathogen or clearing an intracellular pathogen from the circulation.

In one embodiment, the antagonist is an anti-CRIg antibody or a fragment thereof, where the antibody fragment may, for example, be selected from the group consisting of Fab, Fab′, F(ab′)₂, scFv, (scFv)₂, dAb, complementarity determining region (CDR) fragments, linear antibodies, single-chain antibody molecules, minibodies, diabodies, and multispecific antibodies formed from antibody fragments.

In another embodiment, the CRIg antagonist is a monoclonal antibody binding essentially to the same epitope as an anti-CRIg antibody selected from the group consisting of 14G8 (ATCC Deposit No. PTA-8298), 3D10 (ATCC Deposit No. PTA-8299) and 2H1 (ATCC Deposit No. PTA-8300), or a fragment thereof.

In yet another embodiment, the CRIg antagonist is a monoclonal anti-CRIg antibody selected from the group consisting of 14G8 (ATCC Deposit No. PTA-8298), 3D10 (ATCC Deposit No. PTA-8299) and 2H1 (ATCC Deposit No. PTA-8300), or a fragment thereof.

In all embodiments, the antibody or antibody fragment may be chimeric, humanized or human.

In all embodiments, the intracellular pathogen may be selected from the group consisting of viruses, parasites, bacteria, fungi and prions.

In another aspect, the invention concerns nucleic acid encoding an a blocking anti-CRIg antibody herein, or a fragment thereof.

In a different aspect, the invention concerns a composition comprising a CRIg antagonist in admixture with a carrier.

In a further aspect, the invention concerns a method for inhibiting CRIg-mediated complement-dependent cellular entry of an intracellular pathogen, comprising administering to a mammalian subject in need an effective amount of a CRIg antagonist.

In a still further aspect, the invention concerns a method for clearing an intracellular pathogen from the circulation of a mammalian subject, comprising administering to the subject an effective amount of a CRIg antagonist.

In another aspect, the invention concerns a method for the prevention or treatment of an infectious disease comprising administering to a mammalian subject in need an effective amount of a CRIg antagonist.

In all aspects, the mammalian subject preferably is human, and the CRIg antagonist preferably is a blocking anti-CRIg antibody.

In a further aspect, the invention concerns a kit comprising a CRIg antagonist and instructions for administering said CRIg antagonist to treat an infectious disease.

### BRIEF DESCRIPTION OF THE DRAWINGS

FIGS. 1A-1B show the nucleotide and amino acid sequences of the 399-amino acid full-length long form of native human CRIg (huCRIg, SEQ ID NOS: 1 and 2, respectively).

FIGS. 2A-2B show the nucleotide and amino acid sequences of the 305-amino acid short form of native human CRIg (huCRIg-short, SEQ ID NOS: 3 and 4, respectively).

FIGS. 3A-3C show the nucleotide and amino acid sequences of the 280-amino acid native murine CRIg (muCRIg, SEQ ID NOS: 5 and 6, respectively).

FIGS. 4 and 5 show the characterization of hybridoma supernatants for their capacity to block muCRIg-iC3b/C3b or huCRIg-iC3b/C3b binding.

FIG. 6. Anti-CRIg antibodies 2H1 and 14G8 block iC3b binding to murine CRIg-expressing CHO cells.

FIG. 7. Anti-CRIg antibody 2H1 blocks binding of iC3b to CRIg+ peritoneal macrophages.

FIG. 8. Anti-CRIg blocking antibody 2H1 inhibits binding of Listeria Monocytogenes to CRIg+ peritoneal macrophages in vitro.

FIG. 9. CRIg antibody 14G8 inhibits clearance of Listeria Monocylogenes from the circulation.

FIG. 10. Anti-CRIg antibody 14G8 inhibits clearance of Staphylococcus aureus strain M from the circulation leading to decreased bacterial loads in the liver and increased bacterial loads in the heart, spleen and kidney.

FIGS. 11A and B. Role of anti-CRIg antibody 14G8 in antigen-induced arthritis (AIA).

FIG. 12. Binding of anti-CRIg antibody 3D10 to CRIg-expressing cells.

FIG. 13. Anti-CRIg antibody 3D10 blocks iC3b binding to cells expressing human and murine CRIg.

Table 1 provides a summary of the characteristics of anti-CRIg antibodies 3D10, 2H1 and 14G8.

### DETAILED DESCRIPTION OF THE INVENTION

### 1. Definitions

The terms “CRIg,” “PRO362,” “JAM4,” and “STIgMA” are used interchangeably, and refer to native sequence and variant CRIg polypeptides.

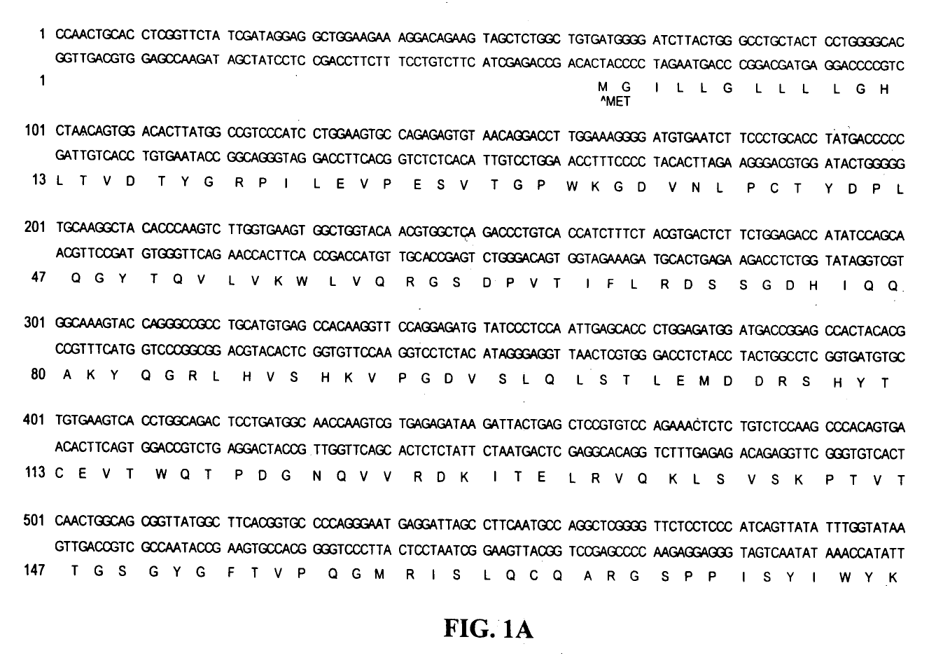

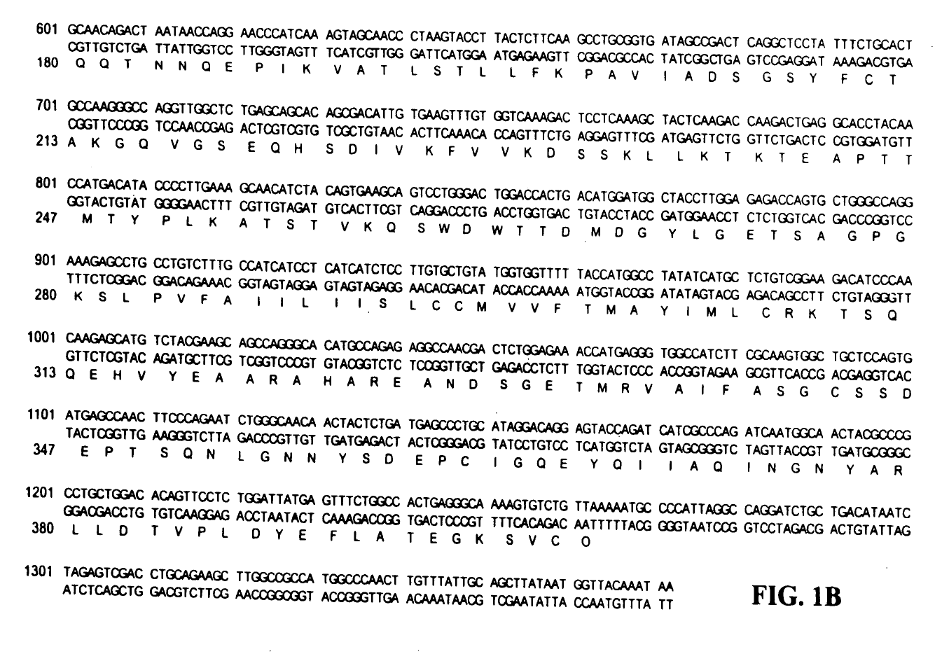

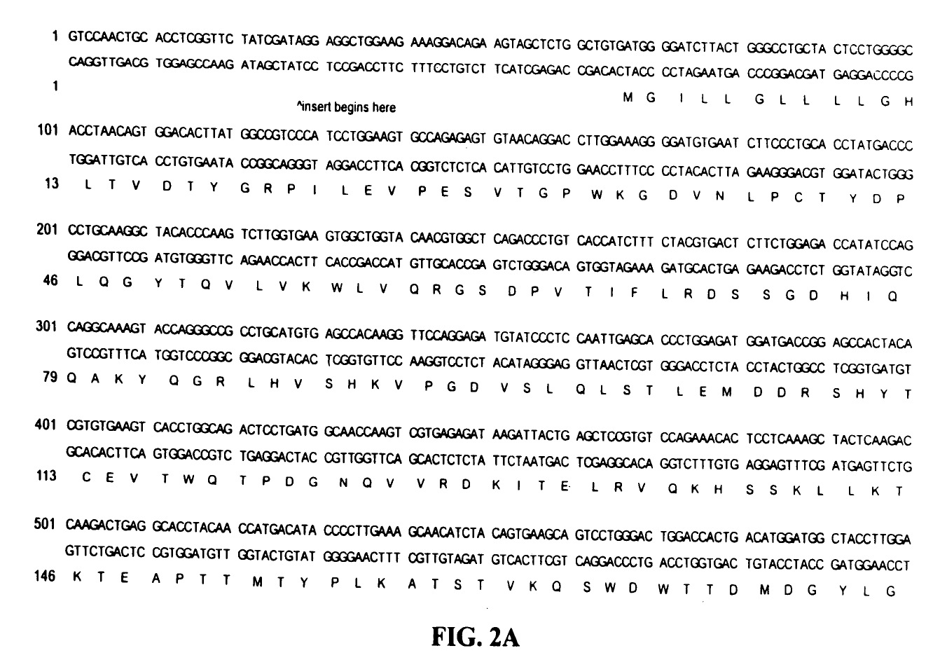

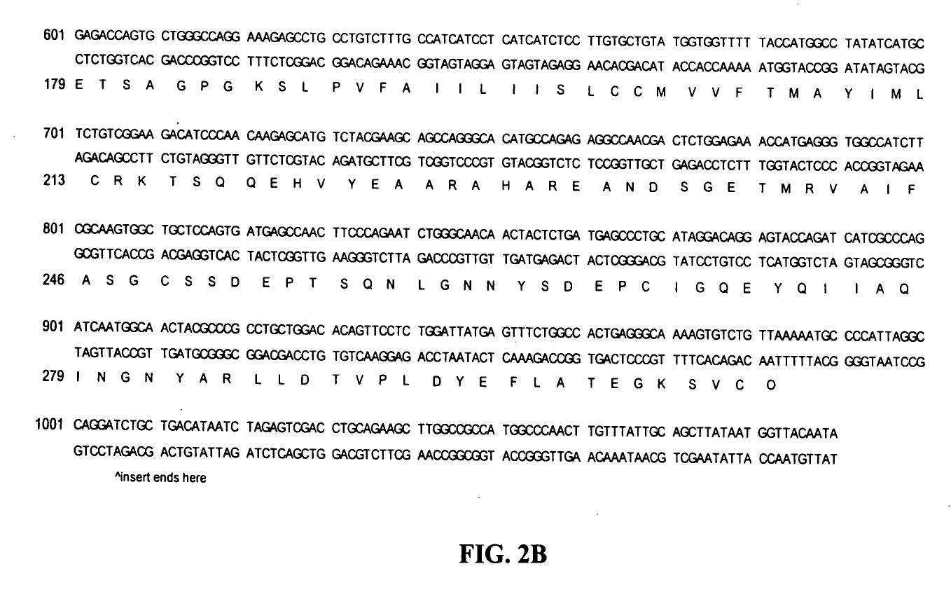

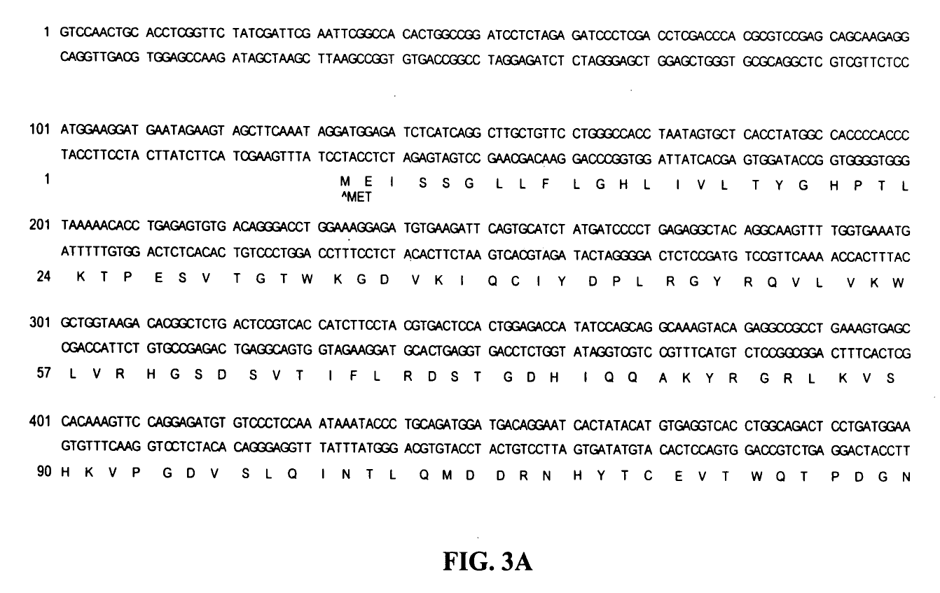

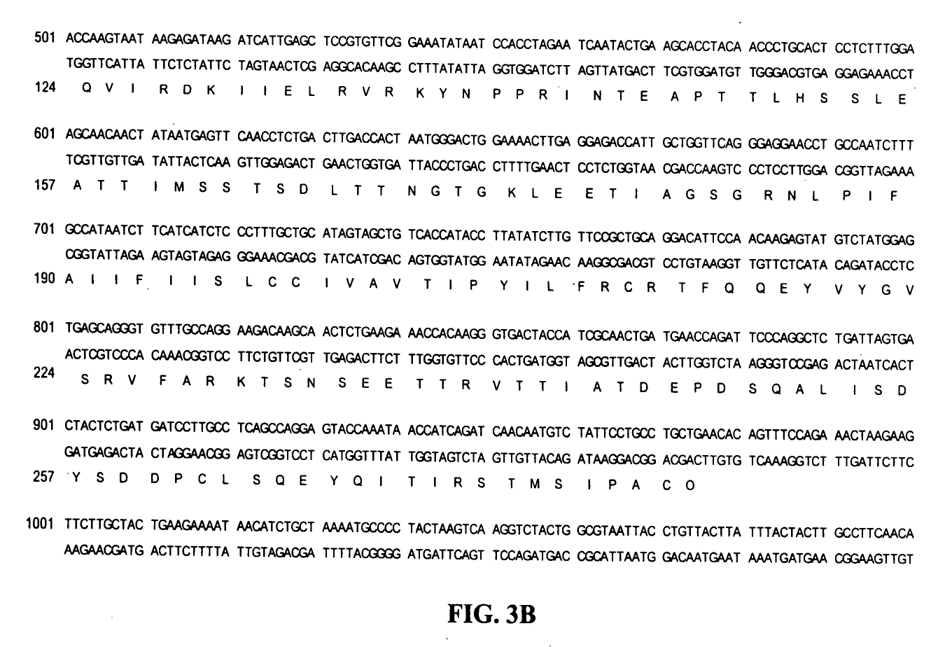

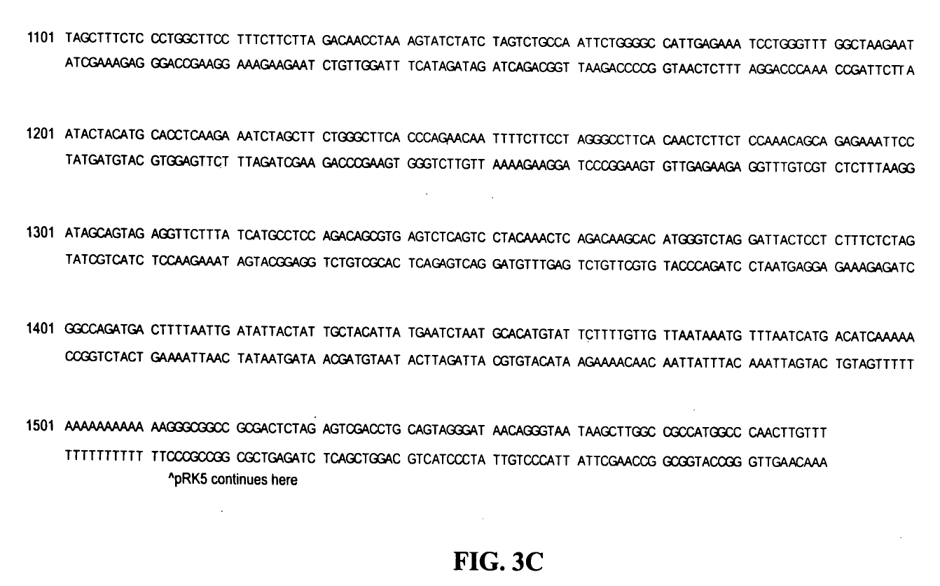

A “native sequence” CRIg, is a polypeptide having the same amino acid sequence as a CRIg polypeptide derived from nature, regardless of its mode of preparation. Thus, native sequence CRIg can be isolated from nature or can be produced by recombinant and/or synthetic means. The term “native sequence CRIg”, specifically encompasses naturally-occurring truncated or secreted forms of CRIg (e.g., an extracellular domain sequence), naturally-occurring variant forms (e.g., alternatively spliced forms) and naturally-occurring allelic variants of CRIg. Native sequence CRIg polypeptides specifically include the full-length 399 amino acids long human CRIg polypeptide of SEQ ID NO: 2 (huCRIg, shown in FIGS. 1A and 1B), with or without an N-terminal signal sequence, with or without the initiating methionine at position 1, and with or without any or all of the transmembrane domain at about amino acid positions 277 to 307 of SEQ ID NO: 2. In a further embodiment, the native sequence CRIg polypeptide is the 305-amino acid, short form of human CRIg (huCRIg-short, SEQ ID NO: 4, shown in FIGS. 2A and 2B), with or without an N-terminal signal sequence, with or without the initiating methionine at position 1, and with or without any or all of the transmembrane domain at about positions 183 to 213 of SEQ ID NO: 4. In a different embodiment, the native sequence CRIg polypeptide is a 280 amino acids long, full-length murine CRIg polypeptide of SEQ ID NO: 6 (muCRIg, shown in FIGS. 3A-3C), with or without an N-terminal signal sequence, with or without the initiating methionine at position I, and with or without any or all of the transmembrane domain at about amino acid positions 181 to 211 of SEQ ID NO: 6. CRIg polypeptides of other non-human animals, including higher primates and mammals, are specifically included within this definition.

The CRIg “extracellular domain” or “ECD” refers to a form of the CRIg polypeptide, which is essentially free of the transmembrane and cytoplasmic domains of the respective full length molecules. Ordinarily, the CRIg ECD will have less than 1% of such transmembrane and/or cytoplasmic domains and preferably, will have less than 0.5% of such domains. CRIg ECD may comprise amino acid residues I or about 21 to X of SEQ ID NO: 2, 4, or 6, where X is any amino acid from about 271 to 281 in SEQ ID NO: 2, any amino acid from about 178 to 186 in SEQ ID NO: 4, and any amino acid from about 176 to 184 in SEQ ID NO: 6.

The term “CRIg antagonist” is used in the broadest sense and includes any molecule that blocks the binding of a native sequence CRIg polypeptide to C3b and/or iC3b, and partially or fully blocks or neutralizes (collectively referred to as “inhibits”) a qualitative biological activity of the native sequence CRIg polypeptide. The biological activity preferably is the ability of CRIg to mediate complement-dependent cellular entry of intracellular pathogens and/or to prevent unwanted clearance of erythrocytes or platelets. Thus, preferred antagonists inhibit CRIg-mediated complement-dependent cellular entry of intracellular pathogens. Suitable CRIg antagonist molecules specifically include, without limitation, blocking anti-CRIg antibodies, including antibody fragments, other polypeptides, such as variants and fusions of the native sequence CRIg polypeptides herein, peptide and non-peptide (organic) small molecules, and antisense polynucleotide molecules. A preferred group of CRIg antagonists includes anti-CRIg antibodies including antibody fragments, specifically binding a native CRIg polypeptide and blocking its binding to C3b and/or iC3b.

A “blocking antibody,” as defined herein is an antibody that blocks the binding of a native sequence CRIg polypeptide to C3b and/or iC3b, and partially or fully blocks or neutralizes (collectively referred to as “inhibits”) a qualitative biological activity of the native sequence CIg polypeptide. The biological activity preferably is the ability of CRIg to mediate complement-dependent cellular entry of intracellular pathogens. Thus, preferred blocking antibodies inhibit CRIg-mediated complement-dependent cellular entry of intracellular pathogens and/or prevent unwanted clearance of erythrocytes or platelets.

A “small molecule” is defined herein to have a molecular weight below about 600, preferably below about 1000 daltons, such as, for example, between 100 and 600, or 100 and 1000 daltons. The term “non-peptide small molecule” refers to small organic compounds in the indicated molecular weight range.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas. The term “antibody” is used in the broadest sense and specifically covers, without limitation, intact monoclonal antibodies, polyclonal antibodies, multispecific antibodies (e.g. bispecific antibodies) formed from at least two intact antibodies, and antibody fragments so long as they exhibit the desired biological activity.

“Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., NIH Publ. No. 91-3242, Vol. 1, pages 647-669 (1991)). The constant domains are not involved directly in binding an antibody to an antigen, but exhibit various effector functions, such as participation of the antibody in antibody-dependent cellular toxicity.

“Antibody fragments” comprise a portion of an intact antibody, preferably the antigen binding or variable region of the intact antibody. Examples of antibody fragments include, but are not limited to, Fab, Fab′, F(ab′)₂, scFv, (scFv)₂, dAb, and complementarity determining region (CDR) fragments, linear antibodies, single-chain antibody molecules, minibodies, diabodies, multispecific antibodies formed from antibody fragments, and, in general, polypeptides that contain at least a portion of an immunoglobulin that is sufficient to confer specific antigen binding to the polypeptide.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called γ, μ, δ, α, and ε, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222: 581-597 (1991), for example. See also U.S. Pat. Nos. 5,750,373, 5,571,698, 5,403,484 and 5,223,409 which describe the preparation of antibodies using phagemid and phage vectors.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)).

“Humanized” forms of non-human (e.g., murine) antibodies are chimeric immunoglobulins, immunoglobulin chains or fragments thereof, which contain minimal sequence derived from non-human immunoglobulin. For the most part, humanized antibodies are human immunoglobulins (recipient antibody) in which several or all residues from a complementarity-determining region (CDR) of the recipient are replaced by residues from a CDR of a non-human species (donor antibody) such as mouse, rat or rabbit having the desired specificity, affinity, and capacity. In some instances, certain Fv framework region (FR) residues of the human immunoglobulin can also be replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525 (1986); Reichmann et al., Nature, 332: 323-329 [1988]; and Presta, Curr. Op. Struct. Biol., 2: 593-596 (1992). The humanized antibody includes a “primatized” antibody where the antigen-binding region of the antibody is derived from an antibody produced by immunizing macaque monkeys with the antigen of interest. Antibodies containing residues from Old World monkeys are also possible within the invention. See, for example, U.S. Pat. Nos. 5,658,570; 5,693,780; 5,681,722; 5,750,105; and 5,756,096.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90: 6444-6448 (1993).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The term “infectious disease” is used herein to refer to any disease that is caused by an infectious organism. Infectious organisms may comprise viruses, (e.g., single stranded RNA viruses, single stranded DNA viruses, human immunodeficiency virus (HIV), hepatitis A, B, and C virus, herpes simplex virus (HSV), cytomegalovirus (CMV) Epstein-Barr virus (EBV), human papilloma virus (HPV)), parasites (e.g., protozoan and metazoan pathogens such as Plasmodia species, Leishmania species, e.g. Leishmania major, Schistosoma species, Trypanosoma species), bacteria (e.g., Mycobacteria, in particular, M. tuberculosis, M. leprae, Yersinia pseudotuberculosis, Salmonella typhimurium, Listeria Monocytogenes, Streptococci, E. coli, Staphylococci), fungi (e.g., Candida species, Aspergillus species), Pneumocystis carinii, and prions.

“Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented.

The term “preventing or inhibiting the development of an infectious disease,” as used herein, refers to the situation wherein the occurrence of the infectious disease is prevented or the onset of the infectious disease is delayed, or the spread of an existing infection is reversed.

“Ameliorate” as used herein, is defined herein as to make better or improve.

The term “mammal” as used herein refers to any animal classified as a mammal, including, without limitation, humans, non-human primates, domestic and farm animals, and zoo, sports or pet animals such horses, pigs, cattle, dogs, cats and ferrets, etc. In a preferred embodiment of the invention, the mammal is a higher primate, most preferably human.

### II. Detailed Description

### Identification of Antagonists of CRIg polypeptides

Antagonists of CRIg polypeptides are molecules that block the binding of a native sequence CRIg polypeptide to C3b and/or iC3b, and partially or fully block, inhibit, or neutralize a qualitative biological activity of the native sequence CRIg polypeptide. CRIg antagonists include, but are not limited to, anti-CRIg antibodies (including antibody fragments), polypeptides, peptide and non-peptide small molecules, and antisense molecules, and can be identified by methods known in the art, including standard binding and/or functional assays. Antagonists of native sequence CRIg polypeptides find utility in the prevention and treatment of infectious diseases due to their ability to prevent the cellular entry of intracellular pathogens, such as viruses, bacteria or parasites.

Anti-CRIg antibodies can be produced by methods known in the art, such as those described hereinabove and in Example 1. Other molecules with the ability to bind CRIg can be identified in binding assays well known in the art.

All binding assays for antagonists are common in that they call for contacting the candidate antagonist with a CRIg polypeptide under conditions and for a time sufficient to allow these two components to interact. In binding assays, the interaction is binding, and the complex formed can be isolated or detected in the reaction mixture. In a particular embodiment, either the CRIg polypeptide or the candidate antagonist is immobilized on a solid phase, e.g., on a microtiter plate, by covalent or non-covalent attachments. Non-covalent attachment generally is accomplished by coating the solid surface with a solution of the CRIg polypeptide and drying. Alternatively, an immobilized antibody, e.g., a monoclonal antibody, specific for the CRIg polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound is a polypeptide which interacts with but does not bind to CRIg, the interaction of CRIg with the respective polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

It is emphasized that the screening assays specifically discussed herein are for illustration only. A variety of other assays, which can be selected depending on the type of the antagonist candidates screened (e.g. polypeptides, peptides, non-peptide small organic molecules, nucleic acid, etc.) are well know to those skilled in the art and are equally suitable for the purposes of the present invention.

The assays described herein may be used to screed libraries of compounds, including, without limitation, chemical libraries, natural product libraries (e.g. collections of microorganisms, animals, plants, etc.), and combinatorial libraries comprised of random peptides, oligonucleotides or small organic molecules. In a particular embodiment, the assays herein are used to screen antibody libraries, including, without limitation, naïve human, recombinant, synthetic and semi-synthetic antibody libraries. The antibody library can, for example, be a phage display library, including monovalent libraries, displaying on average one single-chain antibody or antibody fragment per phage particle, and multi-valent libraries, displaying, on average, two or more antibodies or antibody fragments per viral particle. However, the antibody libraries to be screened in accordance with the present invention are not limited to phage display libraries. Other display technique include, for example, ribosome or mRNA display (Mattheakis et al., Proc. Natl. Acad. Sci. USA 91:9022-9026 (1994); Hanes and Pluckthun, Proc. Natl. Acad. Sci. USA 94:4937-4942 (1997)), microbial cell display, such as bacterial display (Georgiou et al., Nature Biotech. 15:29-34 (1997)), or yeast cell display (Kieke et al., Protein Eng. 10:1303-1310 (1997)), display on mammalian cells, spore display, viral display, such as retroviral display (Urban et al., Nucleic Acids Res. 33:e35 (2005), display based on protein-DNA linkage (Odegrip et al., Proc. Acad. Natl. Sci. USA 101:2806-2810 (2004); Reiersen et al., Nucleic Acids Res. 33:e10 (2005)), and microbead display (Sepp et al., FEBS Lett. 532:455-458 (2002)). Libraries of other molecules, such as combinatorial libraries of synthetic small molecules can also be screened in a similar manner.

The ability of candidate antagonists to block binding of CRIg to C3b and/or iC3b can be assessed in cell-based assays. Thus, CRIg-expressing cells, such as recombinant CHO cells transfected with and expressing CRIg nucleic acid, can be incubated with C3b/iC3b and one or more candidate antagonists, which may or may not have been shown to bind CRIg in previous experiments, and the results analyzed by methods known in the art, such as FACScan, as described in Example 2. Alternatively or in addition, the ability of candidate antagonists to block the binding of CRIg to C3b and/or iC3b can be tested in assays using cells naturally expressing CRIg, such as peritoneal macrophages.

The results obtained in the primary binding/interaction assays herein can be confirmed and supplemented in in vitro and/or in vivo assays of various infections. Alternatively, in vitro and/or in vivo assays of efficacy in the treatment of infections or infectious diseases may be used as primary assays to identify the CRIg antagonists herein. Thus, inhibition of the binding of infectious agents, such as Lysteria monocytogenes, to CRIg-expressing peritoneal macrophages in vivo can be tested as described in Example 2.

Animal models can also be used to test the antibodies and other CRIg antagonists herein. Inhibition of the clearance of Listeria and Staphylococcus aureus from the circulation of mice is described in Example 2.

### Preparation of Anti-CRIg Antagonist Antibodies

(i) Antigen Preparation

Soluble antigens or fragments thereof, optionally conjugated to other molecules, can be used as immunogens for generating antibodies. For transmembrane molecules, such as receptors, fragments of these (e.g. the extracellular domain of a receptor) can be used as the immunogen. Alternatively, cells expressing the transmembrane molecule can be used as the immunogen. Such cells can be derived from a natural source (e.g. cancer cell lines) or may be cells which have been transformed by recombinant techniques to express the transmembrane molecule. Other antigens and forms thereof useful for preparing antibodies will be apparent to those in the art.

(ii) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R₁N═C═NR, where R and R₁ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(iii) Monoclonal Antibodies

Monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al, Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In a further embodiment, antibodies or antibody fragments can be isolated from antibody phage libraries generated using the techniques described in McCafferty et al., Nature, 348:552-554 (1990).

Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iv) Humanized and Human Antibodies

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al, Proc. Natl. Acad. Sci. USA, 90:2551 (1993); Jakobovits et al., Nature, 362:255-258 (1993); Bruggermann et al., Year in Immunol., 7:33 (1993); and Duchosal et al. Nature 355:258 (1992). Human antibodies can also be derived from phage-display libraries (Hoogenboom et al, J. Mol. Biol., 227:381 (1991); Marks et al, J. Mol. Biol., 222:581-597 (1991); Vaughan et al. Nature Biotech 14:309 (1996)). Generation of human antibodies from antibody phage display libraries is further described below.

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). In another embodiment as described in the example below, the F(ab′)₂ is formed using the leucine zipper GCN4 to promote assembly of the F(ab′)₂ molecule. According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

(vi) Multispecific Antibodies

Multispecific antibodies have binding specificities for at least two different epitopes, where the epitopes are usually from different antigens. While such molecules normally will only bind two different epitopes (i.e. bispecific antibodies, BsAbs), antibodies with additional specificities such as trispecific antibodies are encompassed by this expression when used herein. Examples of BsAbs include those with one arm directed against CRIg and another arm directed against another protein playing a role in immune complex clearance, such as a macrophage receptor selected from the group of CR1, CR2, CR3, and CR4.

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991). According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′-SH fragments can also be directly recovered from E. coli, and can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al, J. Immunol. 152:5368 (1994).

Antibodies with more than two specificities are contemplated. For example, trispecific antibodies can be prepared. Tuft et al. J. Immunol. 147: 60 (1991). In addition, multivalent (e.g. bivalent) antibodies with more than one binding specificity to the same antigen are also within the scope herein.

(vii) Effector Function Engineering

It may be desirable to modify the antibody of the invention with respect to effector function, so as to enhance the effectiveness of the antibody. For example cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctonal cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al Anti-Cancer Drug Design 3:219-230 (1989).

(viii) Antibody-Salvage Receptor Binding Epitope Fusions.

In certain embodiments of the invention, it may be desirable to use an antibody fragment, rather than an intact antibody, to increase tumor penetration, for example. In this case, it may be desirable to modify the antibody fragment in order to increase its serum half life. This may be achieved, for example, by incorporation of a salvage receptor binding epitope into the antibody fragment (e.g. by mutation of the appropriate region in the antibody fragment or by incorporating the epitope into a peptide tag that is then fused to the antibody fragment at either end or in the middle, e.g., by DNA or peptide synthesis).

The salvage receptor binding epitope preferably constitutes a region wherein any one or more amino acid residues from one or two loops of a Fc domain are transferred to an analogous position of the antibody fragment. Even more preferably, three or more residues from one or two loops of the Fc domain are transferred. Still more preferred, the epitope is taken from the CH2 domain of the Fc region (e.g., of an IgG) and transferred to the CH1, CH3, or V.sub.H region, or more than one such region, of the antibody. Alternatively, the epitope is taken from the CH2 domain of the Fc region and transferred to the CL region or VL region, or both, of the antibody fragment.

(ix) Other Covalent Modifications of Antibodies

Covalent modifications of antibodies are included within the scope of this invention. They may be made by chemical synthesis or by enzymatic or chemical cleavage of the antibody, if applicable. Other types of covalent modifications of the antibody are introduced into the molecule by reacting targeted amino acid residues of the antibody with an organic derivatizing agent that is capable of reacting with selected side chains or the N- or C-terminal residues. Examples of covalent modifications are described in U.S. Pat. No. 5,534,615, specifically incorporated herein by reference. A preferred type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

(x) Generation of Antibodies From Synthetic Antibody Phage Libraries

In a preferred embodiment, the invention provides a method for generating and selecting novel antibodies using a unique phage display approach. The approach involves generation of synthetic antibody phage libraries based on single framework template, design of sufficient diversities within variable domains, display of polypeptides having the diversified variable domains, selection of candidate antibodies with high affinity to target the antigen, and isolation of the selected antibodies.

Details of the phage display methods can be found, for example, WO03/102157 published Dec. 11, 2003, the entire disclosure of which is expressly incorporated herein by reference.

In one aspect, the antibody libraries used in the invention can be generated by mutating the solvent accessible and/or highly diverse positions in at least one CDR of an antibody variable domain. Some or all of the CDRs can be mutated using the methods provided herein. In some embodiments, it may be preferable to generate diverse antibody libraries by mutating positions in CDRH1, CDRH2 and CDRH3 to form a single library or by mutating positions in CDRL3 and CDRH3 to form a single library or by mutating positions in CDRL3 and CDRH1, CDRH2 and CDRH3 to form a single library.

A library of antibody variable domains can be generated, for example, having mutations in the solvent accessible and/or highly diverse positions of CDRH1, CDRH2 and/or CDRH3. Another library can be generated having mutations in CDRH1, CDRH2 and/or CDRH3. These libraries can also be used in conjunction with each other to generate binders of desired affinities. For example, after one or more rounds of selection of heavy chain libraries for binding to a target antigen, a light chain library can be replaced into the population of heavy chain binders for further rounds of selection to increase the affinity of the binders.

Preferably, a library is created by substitution of original amino acids with variant amino acids in the CDRH3 region of the variable region of the heavy chain sequence. The resulting library can contain a plurality of antibody sequences, wherein the sequence diversity is primarily in the CDRH3 region of the heavy chain sequence.

In one aspect, the library is created in the context of the humanized antibody 4D5 sequence, or the sequence of the framework amino acids of the humanized antibody 4D5 sequence. Preferably, the library is created by substitution of at least residues 95-100a of the heavy chain with amino acids encoded by the DVK codon set, wherein the DVK codon set is used to encode a set of variant amino acids for every one of these positions. An example of an oligonucleotide set that is useful for creating these substitutions comprises the sequence (DVK)₇. In some embodiments, a library is created by substitution of residues 95-100a with amino acids encoded by both DVK and NNK codon sets. An example of an oligonucleotide set that is useful for creating these substitutions comprises the sequence (DVK)₆ (NNK). In another embodiment, a library is created by substitution of at least residues 95-100a with amino acids encoded by both DVK and NNK codon sets. An example of an oligonucleotide set that is useful for creating these substitutions comprises the sequence (DVK)₅ (NNK). Another example of an oligonucleotide set that is useful for creating these substitutions comprises the sequence (NNK)₆. Other examples of suitable oligonucleotide sequences can be determined by one skilled in the art according to the criteria described herein.

In another embodiment, different CDRH3 designs are utilized to isolate high affinity binders and to isolate binders for a variety of epitopes. The range of lengths of CDRH3 generated in this library is 11 to 13 amino acids, although lengths different from this can also be generated. H3 diversity can be expanded by using NNK, DVK and NVK codon sets, as well as more limited diversity at N and/or C-terminal.

Diversity can also be generated in CDRH1 and CDRH2. The designs of CDR-H1 and H2 diversities follow the strategy of targeting to mimic natural antibodies repertoire as described with modification that focus the diversity more closely matched to the natural diversity than previous design.

For diversity in CDRH3, multiple libraries can be constructed separately with different lengths of H3 and then combined to select for binders to target antigens. The multiple libraries can be pooled and sorted using solid support selection and solution sorting methods as described previously and herein below. Multiple sorting strategies may be employed. For example, one variation involves sorting on target bound to a solid, followed by sorting for a tag that may be present on the fusion polypeptide (e.g., anti-gD tag) and followed by another sort on target bound to solid. Alternatively, the libraries can be sorted first on target bound to a solid surface, the eluted binders are then sorted using solution phase binding with decreasing concentrations of target antigen. Utilizing combinations of different sorting methods provides for minimization of selection of only highly expressed sequences and provides for selection of a number of different high affinity clones.

High affinity binders for the target antigen can be isolated from the libraries. Limiting diversity in the H1/H2 region decreases degeneracy about 10⁴ to 10⁵ fold and allowing more H3 diversity provides for more high affinity binders. Utilizing libraries with different types of diversity in CDRH3 (e.g. utilizing DVK or NVT) provides for isolation of binders that may bind to different epitopes of a target antigen.

Of the binders isolated from the pooled libraries as described above, it has been discovered that affinity may be further improved by providing limited diversity in the light chain. Light chain diversity is generated in this embodiment as follows in CDRL1: amino acid position 28 is encoded by RDT; amino acid position 29 is encoded by RKT; amino acid position 30 is encoded by RVW; amino acid position 31 is encoded by ANW; amino acid position 32 is encoded by THT; optionally, amino acid position 33 is encoded by CTG; in CDRL2: amino acid position 50 is encoded by KBG; amino acid position 53 is encoded by AVC; and optionally, amino acid position 55 is encoded by GMA; in CDRL3: amino acid position 91 is encoded by TMT or SRT or both; amino acid position 92 is encoded by DMC; amino acid position 93 is encoded by RVT; amino acid position 94 is encoded by NHT; and amino acid position 96 is encoded by TWT or YKG or both.

In another embodiment, a library or libraries with diversity in CDRH1, CDRH2 and CDRH3 regions is generated. In this embodiment, diversity in CDRH3 is generated using a variety of lengths of H3 regions and using primarily codon sets XYZ and NNK or NNS. Libraries can be formed using individual oligonucleotides and pooled or oligonucleotides can be pooled to form a subset of libraries. The libraries of this embodiment can be sorted against target bound to solid. Clones isolated from multiple sorts can be screened for specificity and affinity using ELISA assays. For specificity, the clones can be screened against the desired target antigens as well as other nontarget antigens. Those binders to the target antigen can then be screened for affinity in solution binding competition ELISA assay or spot competition assay. High affinity binders can be isolated from the library utilizing XYZ codon sets prepared as described above. These binders can be readily produced as antibodies or antigen binding fragments in high yield in cell culture.

In some embodiments, it may be desirable to generate libraries with a greater diversity in lengths of CDRH3 region. For example, it may be desirable to generate libraries with CDRH3 regions ranging from about 7 to 19 amino acids.

High affinity binders isolated from the libraries of these embodiments are readily produced in bacterial and eukaryotic cell culture in high yield. The vectors can be designed to readily remove sequences such as gD tags, viral coat protein component sequence, and/or to add in constant region sequences to provide for production of full length antibodies or antigen binding fragments in high yield.

A library with mutations in CDRH3 can be combined with a library containing variant versions of other CDRs, for example CDRL1, CDRL2, CDRL3, CDRH1 and/or CDRH2. Thus, for example, in one embodiment, a CDRH3 library is combined with a CDRL3 library created in the context of the humanized 4D5 antibody sequence with variant amino acids at positions 28, 29, 30, 31, and/or 32 using predetermined codon sets. In another embodiment, a library with mutations to the CDRH3 can be combined with a library comprising variant CDRH1 and/or CDRH2 heavy chain variable domains. In one embodiment, the CDRH1 library is created with the humanized antibody 4D5 sequence with variant amino acids at positions 28, 30, 31, 32 and 33. A CDRH2 library may be created with the sequence of humanized antibody 4D5 with variant amino acids at positions 50, 52, 53, 54, 56 and 58 using the predetermined codon sets.

(xi) Antibody Mutants

The novel antibodies generated from phage libraries can be further modified to generate antibody mutants with improved physical, chemical and or biological properties over the parent antibody. Where the assay used is a biological activity assay, the antibody mutant preferably has a biological activity in the assay of choice which is at least about 10 fold better, preferably at least about 20 fold better, more preferably at least about 50 fold better, and sometimes at least about 100 fold or 200 fold better, than the biological activity of the parent antibody in that assay. For example, an anti-CRIg antibody mutant preferably has a binding affinity for CRIg which is at least about 10 fold stronger, preferably at least about 20 fold stronger, more preferably at least about 50 fold stronger, and sometimes at least about 100 fold or 200 fold stronger, than the binding affinity of the parent antibody.

To generate the antibody mutant, one or more amino acid alterations (e.g. substitutions) are introduced in one or more of the hypervariable regions of the parent antibody. Alternatively, or in addition, one or more alterations (e.g. substitutions) of framework region residues may be introduced in the parent antibody where these result in an improvement in the binding affinity of the antibody mutant for the antigen from the second mammalian species. Examples of framework region residues to modify include those which non-covalently bind antigen directly (Amit et al. (1986) Science 233:747-753); interact with/effect the conformation of a CDR (Chothia et al. (1987) J. Mol. Biol. 196:901-917); and/or participate in the V_{L}-V_{H} interface (EP 239 400B1). In certain embodiments, modification of one or more of such framework region residues results in an enhancement of the binding affinity of the antibody for the antigen from the second mammalian species. For example, from about one to about five framework residues may be altered in this embodiment of the invention. Sometimes, this may be sufficient to yield an antibody mutant suitable for use in preclinical trials, even where none of the hypervariable region residues have been altered. Normally, however, the antibody mutant will comprise additional hypervariable region alteration(s).

The hypervariable region residues which are altered may be changed randomly, especially where the starting binding affinity of the parent antibody is such that such randomly produced antibody mutants can be readily screened.

One useful procedure for generating such antibody mutants is called “alanine scanning mutagenesis” (Cunningham and Wells (1989) Science 244:1081-1085). Here, one or more of the hypervariable region residue(s) are replaced by alanine or polyalanine residue(s) to affect the interaction of the amino acids with the antigen from the second mammalian species. Those hypervariable region residue(s) demonstrating functional sensitivity to the substitutions then are refined by introducing further or other mutations at or for the sites of substitution. Thus, while the site for introducing an amino acid sequence variation is predetermined, the nature of the mutation per se need not be predetermined. The ala-mutants produced this way are screened for their biological activity as described herein.

Normally one would start with a conservative substitution such as those shown below under the heading of “preferred substitutions”. If such substitutions result in a change in biological activity (e.g. binding affinity), then more substantial changes, denominated “exemplary substitutions” in the following table, or as further described below in reference to amino acid classes, are introduced and the products screened.

### Preferred Substitutions:

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; met; ala; | ile |
| | phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

Even more substantial modifications in the antibodies biological properties are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr, asn, gin;

(3) acidic: asp, glu;

(4) basic: his, lys, arg;

(5) residues that influence chain orientation: gly, pro; and

(6) aromatic: trp, tyr, phe.

Non-conservative substitutions will entail exchanging a member of one of these classes for another class.

In another embodiment, the sites selected for modification are affinity matured using phage display (see above).

Nucleic acid molecules encoding amino acid sequence mutants are prepared by a variety of methods known in the art. These methods include, but are not limited to, oligonucleotide-mediated (or site-directed) mutagenesis, PCR mutagenesis, and cassette mutagenesis of an earlier prepared mutant or a non-mutant version of the parent antibody. The preferred method for making mutants is site directed mutagenesis (see, e.g., Kunkel (1985) Proc. Natl. Acad. Sci. USA 82:488).

In certain embodiments, the antibody mutant will only have a single hypervariable region residue substituted. In other embodiments, two or more of the hypervariable region residues of the parent antibody will have been substituted, e.g. from about two to about ten hypervariable region substitutions.

Ordinarily, the antibody mutant with improved biological properties will have an amino acid sequence having at least 75% amino acid sequence identity or similarity with the amino acid sequence of either the heavy or light chain variable domain of the parent antibody, more preferably at least 80%, more preferably at least 85%, more preferably at least 90%, and most preferably at least 95%. Identity or similarity with respect to this sequence is defined herein as the percentage of amino acid residues in the candidate sequence that are identical (i.e. same residue) or similar (i.e. amino acid residue from the same group based on common side-chain properties, see above) with the parent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

Following production of the antibody mutant, the biological activity of that molecule relative to the parent antibody is determined. As noted above, this may involve determining the binding affinity and/or other biological activities of the antibody. In a preferred embodiment of the invention, a panel of antibody mutants is prepared and screened for binding affinity for the antigen or a fragment thereof. One or more of the antibody mutants selected from this initial screen are optionally subjected to one or more further biological activity assays to confirm that the antibody mutant(s) with enhanced binding affinity are indeed useful, e.g. for preclinical studies.

The antibody mutant(s) so selected may be subjected to further modifications, oftentimes depending on the intended use of the antibody. Such modifications may involve further alteration of the amino acid sequence, fusion to heterologous polypeptide(s) and/or covalent modifications such as those elaborated below. With respect to amino acid sequence alterations, exemplary modifications are elaborated above. For example, any cysteine residue not involved in maintaining the proper conformation of the antibody mutant also may be substituted, generally with serine, to improve the oxidative stability of the molecule and prevent aberrant cross linking. Conversely, cysteine bond(s) may be added to the antibody to improve its stability (particularly where the antibody is an antibody fragment such as an Fv fragment). Another type of amino acid mutant has an altered glycosylation pattern. This may be achieved by deleting one or more carbohydrate moieties found in the antibody, and/or adding one or more glycosylation sites that are not present in the antibody. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

(xii) Recombinant Production of Antibodies

For recombinant production of an antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence (e.g. as described in U.S. Pat. No. 5,534,615, specifically incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serrafia, e.g., Serratia marcescans, and Shigeila, as well as Bacilli such as B. subtilis and B. lichenifonnis (e.g., B. licheniformis 41P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X 1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al, J. Gen Virol. 36:59 (1977)); baby hamster kidney cells (BHK, ATCC CCL 10); Chinese hamster ovary cells/−DHFR(CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (WI 38, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce the antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed cells, is removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human .gamma.3 (Guss et al., EMBO J. 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH 3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Uses of CRIg Antagonists

The CRIg antagonists of the present invention, including blocking anti-CRIg antibodies and fragments thereof, can be used to inhibit the cellular entry of intracellular pathogen and/or clearing pathogens from the circulation. Thus, the CRIg antagonists herein can be used to prevent and/or treat infectious diseases caused by such pathogens. Such infectious disease include, without limitation, viral diseases, such as human immunodeficiency virus (HIV), hepatitis A, B, and C virus, herpes simplex virus (HSV), cytomegalovirus (CMV), Epstein-Barr virus (EBV), or human papilloma virus (HPV) infections; diseases caused by parasites, such as Plasmodia species, Leishmania species, e.g. Leishmania major, Schistosoma species, Trypanosoma species; and bacterial infections, such as infections caused by Mycobacteria, in particular, M. tuberculosis, M. leprae, Yersinia pseudotuberculosis, Salmonella typhimurium, Listeria Monocytogenes, Streptococci, E. coli, Staphylococci; fungal infections, such as those caused by Candida species, Aspergillus species, and Pneumocystis carinii.

In addition, the CRIg antagonists of the present invention, including blocking anti-CRIg antibodies and fragments thereof, can be used to prevent unwanted clearance of erythrocytes or platelets through complement receptor CRIg. As a result, the antagonists herein find utility in the prevention or treatment of hemolytic anemia and/or thrombocytopenia.

Therapeutic formulations of the compounds herein are prepared for storage by mixing the compound identified (such as an antibody) having the desired degree of purity with optional physiologically acceptable carriers, excipients or stabilizers (Remington's Pharmaceutical Sciences, supra), in the form of lyophilized cake or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic. polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or PEG.

Compounds to be used for in vivo administration must be sterile. This is readily accomplished by filtration through sterile filtration membranes, prior to or following lyophilization and reconstitution.

Therapeutic compositions may be placed into a container having a sterile access port, for example, an intravenous solution bag or vial having a stopper pierceable by a hypodermic injection needle.

The route of administration is in accord with known methods, e.g. injection or infusion by intravenous, intraperitoneal, intracerebral, intramuscular, intraocular, intraarterial or intralesional routes, topical administration, or by sustained release systems as noted below.

For intracerebral use, the compounds may be administered continuously by infusion into the fluid reservoirs of the CNS, although bolus injection is acceptable. The compounds are preferably administered into the ventricles of the brain or otherwise introduced into the CNS or spinal fluid. Administration may be performed by an indwelling catheter using a continuous administration means such as a pump, or it can be administered by implantation, e.g., intracerebral implantation, of a sustained-release vehicle. More specifically, the compounds can be injected through chronically implanted cannulas or chronically infused with the help of osmotic minipumps. Subcutaneous pumps are available that deliver proteins through a small tubing to the cerebral ventricles. Highly sophisticated pumps can be refilled through the skin and their delivery rate can be set without surgical intervention. Examples of suitable administration protocols and delivery systems involving a subcutaneous pump device or continuous intracerebroventricular infusion through a totally implanted drug delivery system are those used for the administration of dopamine, dopamine agonists, and cholinergic agonists to Alzheimer patients and animal models for Parkinson's disease described by Harbaugh, J. Neural Transm. Suppl., 24:271 (1987); and DeYebenes, et al., Mov. Disord. 2:143 (1987).

Suitable examples of sustained release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman, et al., 1983, Biopolymers 22:547), poly(2-hydroxyethyl-methacrylate) (Langer, et al., 1981, J. Biomed. Mater. Res. 15:167; Langer, 1982, Chem. Tech. 12:98), ethylene vinyl acetate (Langer, et al., Id.) or poly-D-(−)-3-hydroxybutyric acid (EP 133,988A) Sustained release compositions also include liposomally entrapped compounds, which can be prepared by methods known per se. (Epstein, et al., Proc. Natl. Acad. Sci. 82:3688 (1985); Hwang, et al., Proc. Natl. Acad. Sci. USA 77:4030 (1980); U.S. Pat. Nos. 4,485,045 and 4,544,545; and EP 102,324A). Ordinarily the liposomes are of the small (about 200-800 Angstroms) unilamelar type in which the lipid content is greater than about 30 mol. % cholesterol, the selected proportion being adjusted for the optimal therapy.

An effective amount of an active compound to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. A typical daily dosage might range from about 1 μg/kg to up to 100 mg/kg or more, depending on the factors mentioned above. Typically, the clinician will administer an active compound until a dosage is reached that repairs, maintains, and, optimally, reestablishes neuron function. The progress of this therapy is easily monitored by conventional assays.

Further details of the invention are illustrated by the following non-limiting Examples.

### Example 1

Generation of Monoclonal Antibodies to muCRIg and huCRIg-Short

Monoclonal antibodies to murine CRIg (muCRIg) were generated by injecting 2 μg of the immunoadhesin mouse (mu) CRIg-mFc (PUR 9465, mouse CRIg fused to a C-terminal Fc portion of mouse IgG1) in monophosphoryl lipid A/trehalose dicorynomycolate adjuvant (Corixa, Hamilton, Mont.) in the footpads of B6 (KO) mice Jam4 6E3 (Genentech, Inc.), 11 times. Popliteal lymph nodes from mice were fused with P3X63Ag.U.I (ATCC #CRL-1957) myeloma cells. Hybridoma cells were screened against muCRIg-LFH (PUR 9052, mouse CRIg conjugated to a leucine zipper, flag and a (8×) Histidine tag) for binding affinity to muCRIg. Cell lines producing antibodies were cloned by limiting dilution.

In another study, monoclonal antibodies to muCRIg and huCRIg-short were generated by co-immunizing muCRIg-hFc (PUR 9460, mouse CRIg with a C-terminal Fc portion of mouse IgG1) and huCRIg-short-huFc (PUR 10125, human CRIg short form with a C-terminal Fc portion of human IgG1) by injecting 2 μg of each muCRIg and huCRIg-short-huFc in monophosphoryl lipid A/trehalose dicorynomycolate adjuvant (Corixa, Hamilton, Mont.) in the footpads of B6 (KO) mice Jam4 6E3, 11 times. Popliteal nodes from mice were fused with P3X63Ag.U.1 myeloma cells. Hybridoma cells were screened against both muCRIg and huCRIg-short for binding affinity to muCRIg, huCRIg-short, or both. Cell lines producing antibodies were cloned by limiting dilution.

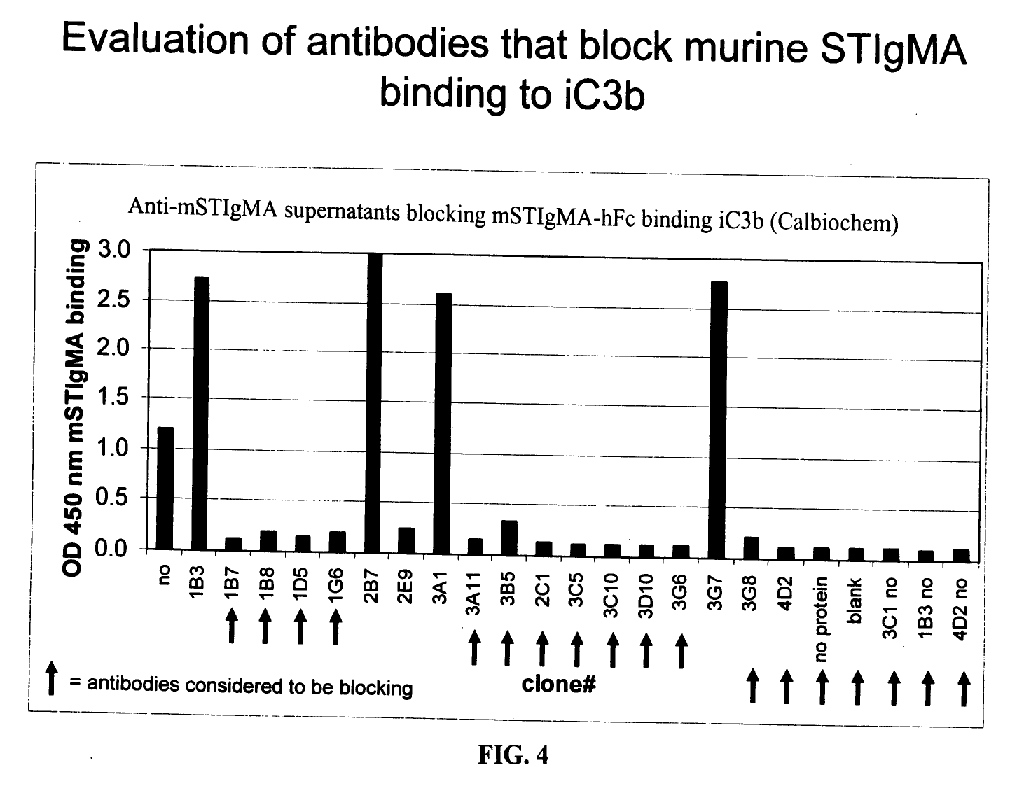

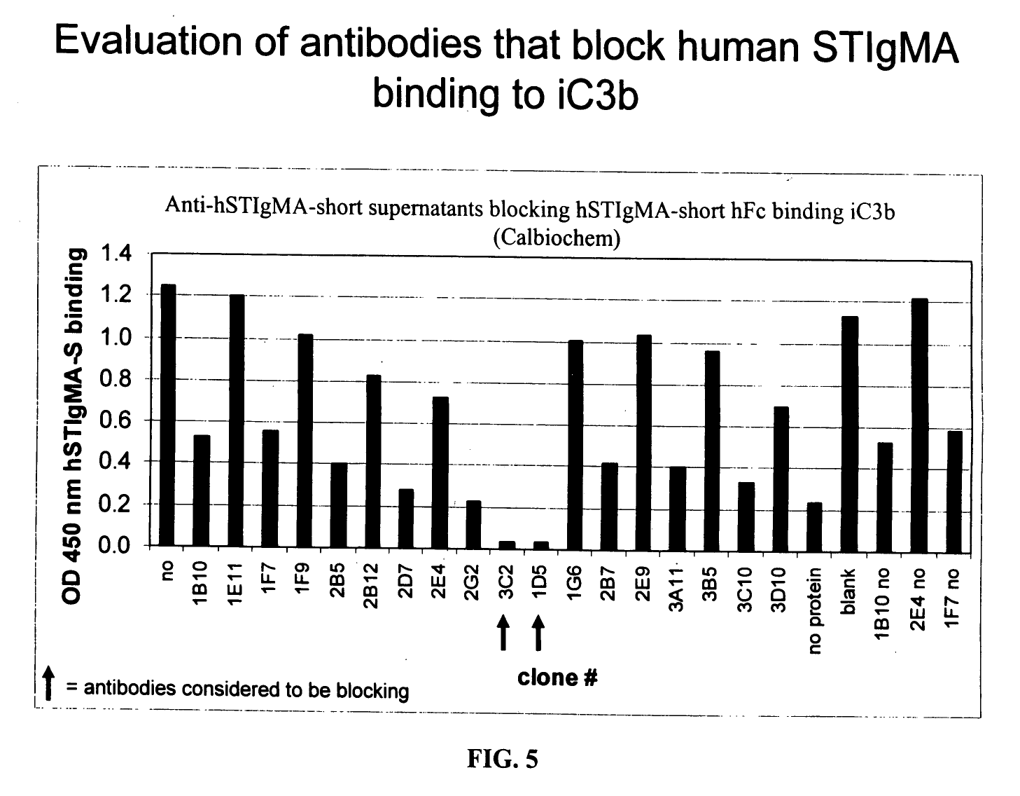

Characterization of hybridoma supernatants for their capacity to block mouse CRIg-iC3b/C3b or human CRIg-iC3b/C3b binding is shown in FIGS. 4 and 5.

### Example 2

### Further Characterization of Monoclonal Antibodies to muCRIg and huCRIg-Short

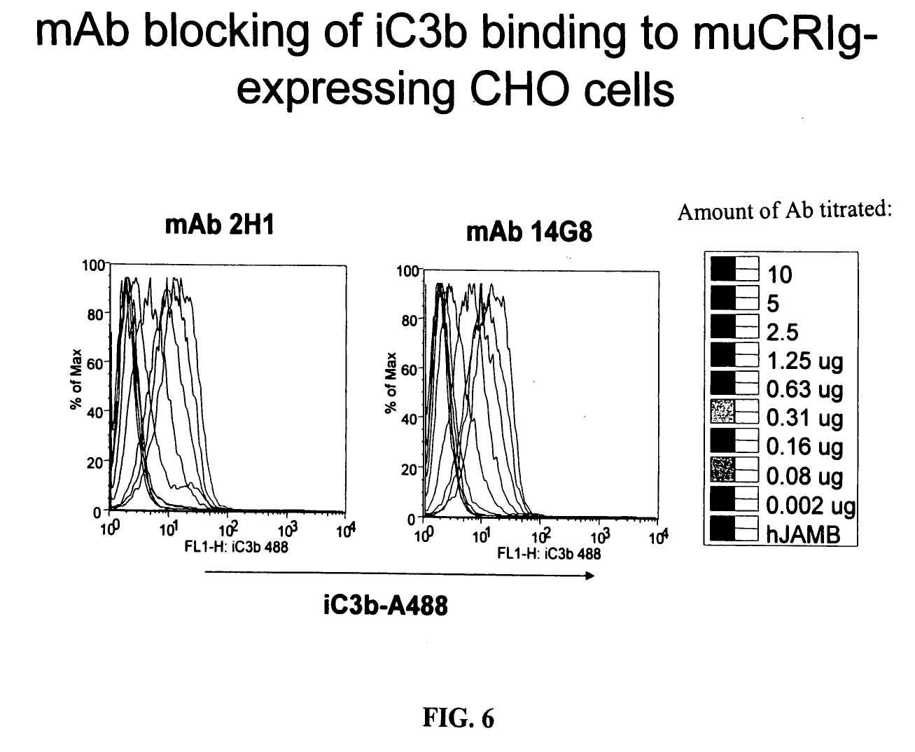

Inhibition of iC3b binding to CRIg-expressing CHO cells incubated with anti CRIg blocking antibodies: 400,000 CHO cells were coated with the antibodies for 25 min at 4° C. and washed 1×. 5 μg/ml (27 nM) iC3b was added for 30 min at 4° C., washed and analyzed on FACScan. The results are shown in FIG. 6.

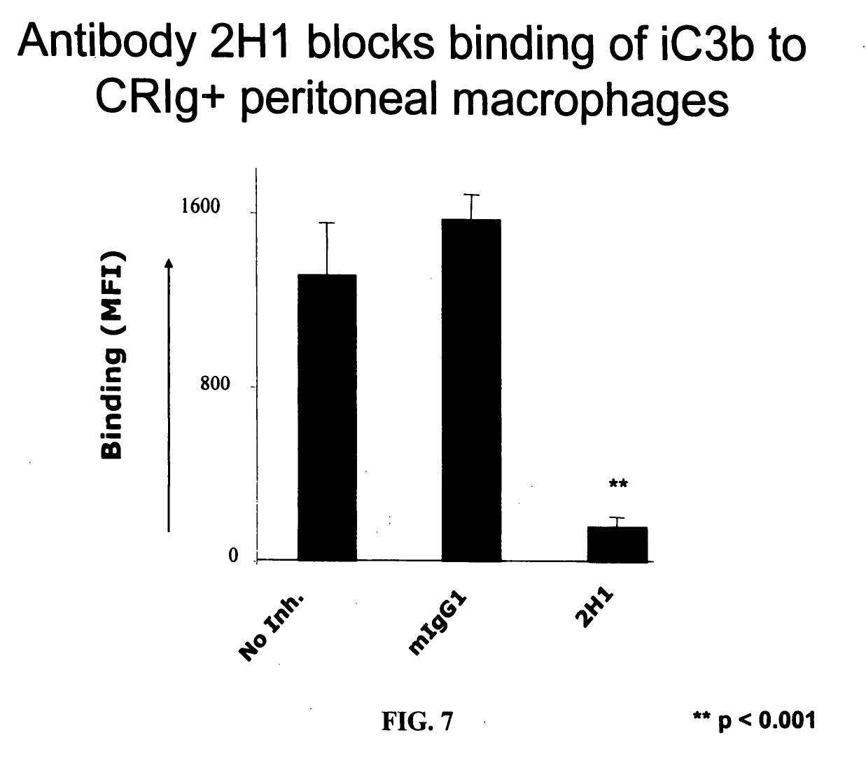

Blocking binding of iC3b to CRIg+ peritoneal macrophages: Peritoneal cells were lavaged, spun once and resuspended in veronal buffer containing 1% gelatin, 1 mM Ca⁺⁺ and 1 mM Mg⁺⁺ (GVB⁺⁺). Cells were incubated with or without endotoxin-free monoclonal antibodies (anti-CRIg mAb, clone 2H1, and isotype control). After incubating for 30 minutes cells were incubated with different concentrations of A488-labeled iC3b (Advanced Research Technologies Inc.). Cells were then blocked for FcR-mediated binding (as above) and stained for 17C9-A647 and F4/80-PE. Binding of complement proteins to CRIg^{high}F4/80^{high} and CRIG^{neg} F4/80^{high} cells was evaluated. The results are shown in FIG. 7.

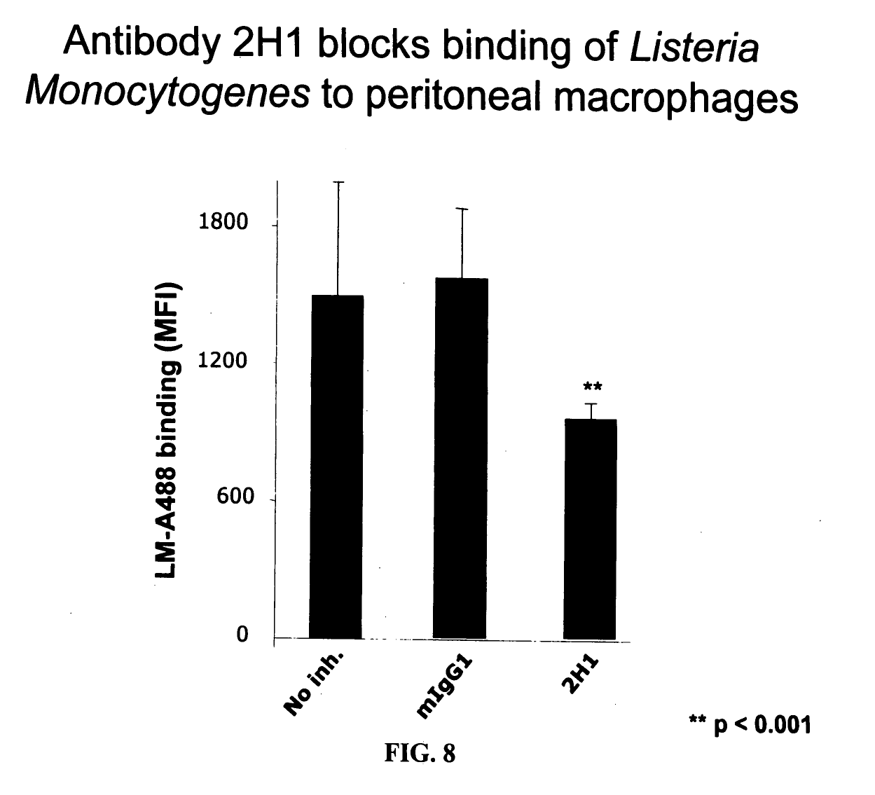

Inhibition of binding of Listeria Monocytogenes to CRIg+ peritoneal macrophages in vitro: Peritoneal cells were lavaged and incubated with or without M1/70 or 2H1 monoclonal antibodies. LM-A488 was suspended in Hank's balanced salt solution (HBSS), mixed with C3 knock out or wild type mouse serum (10%) and rotated at 37° C. for 30 minutes. Serum opsonized-LM-A488 was then added to RPM and mixed on a rocker at 37° C. for 30 minutes. Cells were chilled on ice, blocked for FcR-mediated binding and stained for 17C9-A647 and F4/80-PE. Cells were further incubated with 7-AAD to detect dead cells and debris. After incubating on ice for several minutes cells were washed twice to remove any unbound 7-AAD and antibodies. Cells were suspended in FIBSS and fixed with 1% formaldehyde for 16 hours before running on a 4-color flow cytometry instrument. The results are shown in FIG. 8.

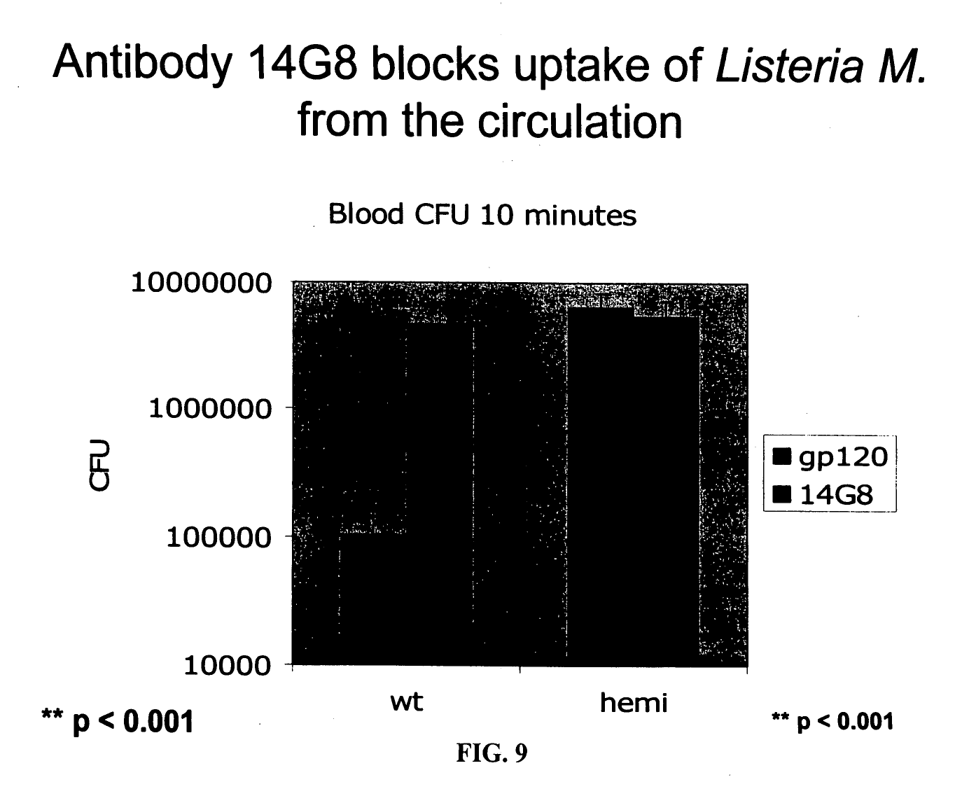

Inhibition of the clearance of Listeria Monocytogenes from the circulation: Mice received two injections of 100 Hg 14G8 blocking antibody the day prior to Listeria M. infection. Injections were 6 hrs apart. One day later, mice were infected with 2×10e7 CFU Listeria Monocytogenes. Blood was drawn 10 minutes later and analyzed for the presence of bacteria by counting colonies grown overnight on brain-heart infusion plates. The results are shown in FIG. 9.

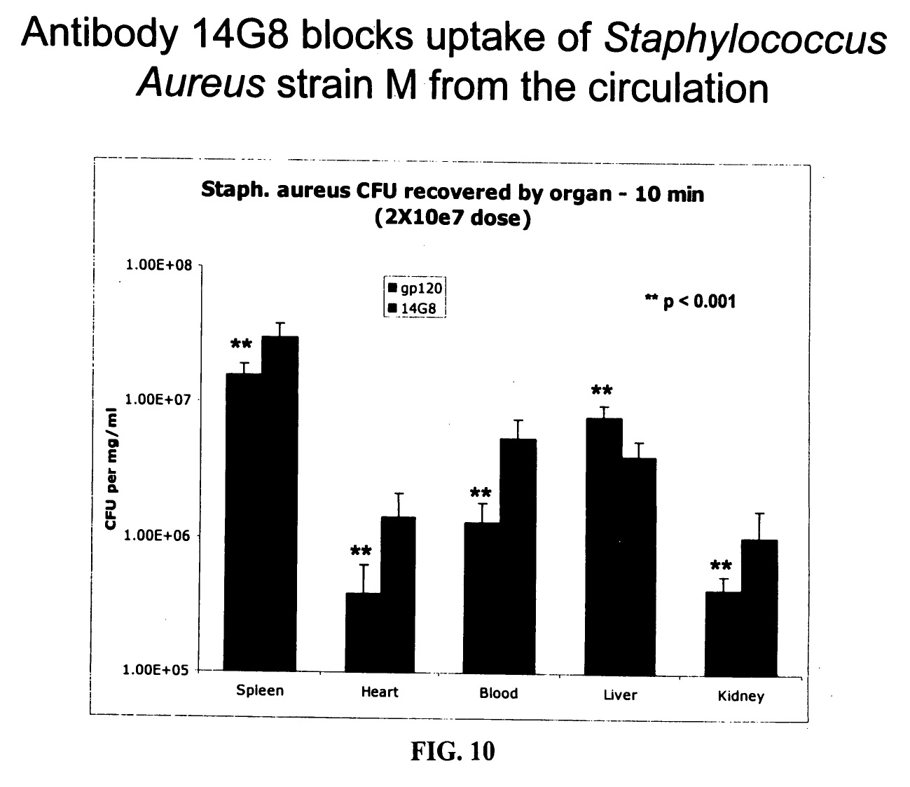

Inhibition of the clearance of Staphylococcus aureus strain M from the circulation: The method used in this experiment was as described above, except that instead of Listeria monocytogenes 2×10e7 CFU Staphylococcus Aureus strain M was injected i.v. The results set forth in FIG. 10 show that the tested CRIg blocking antibody inhibits clearance of Staphylococcus aureus strain M from the circulation leading to decreased bacterial loads in the liver and increased bacterial loads in the heart, spleen and kidney.

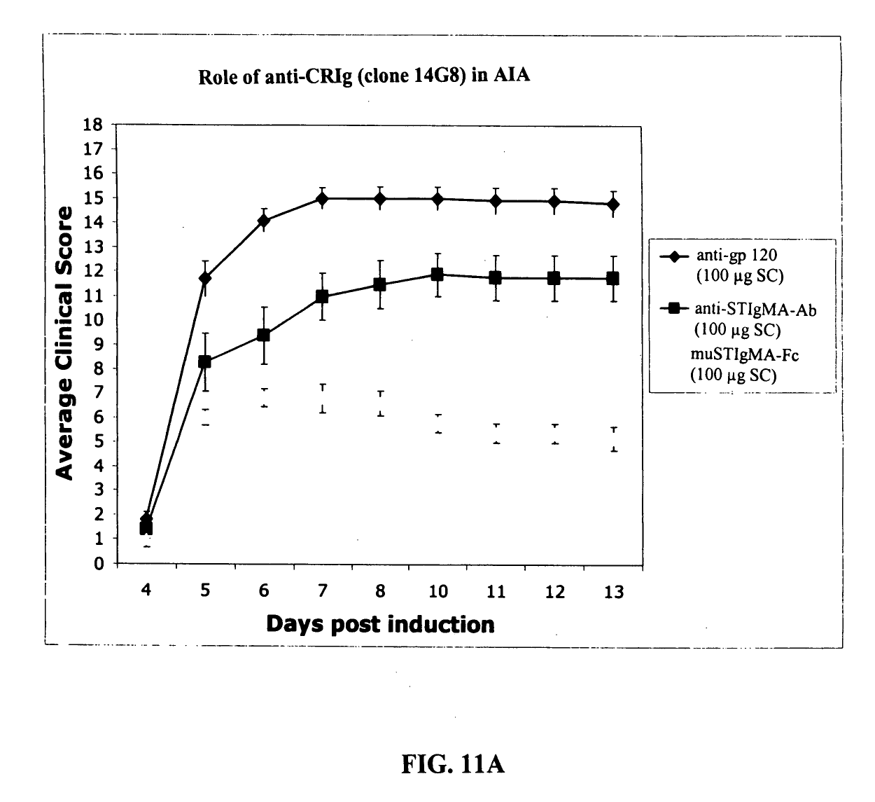

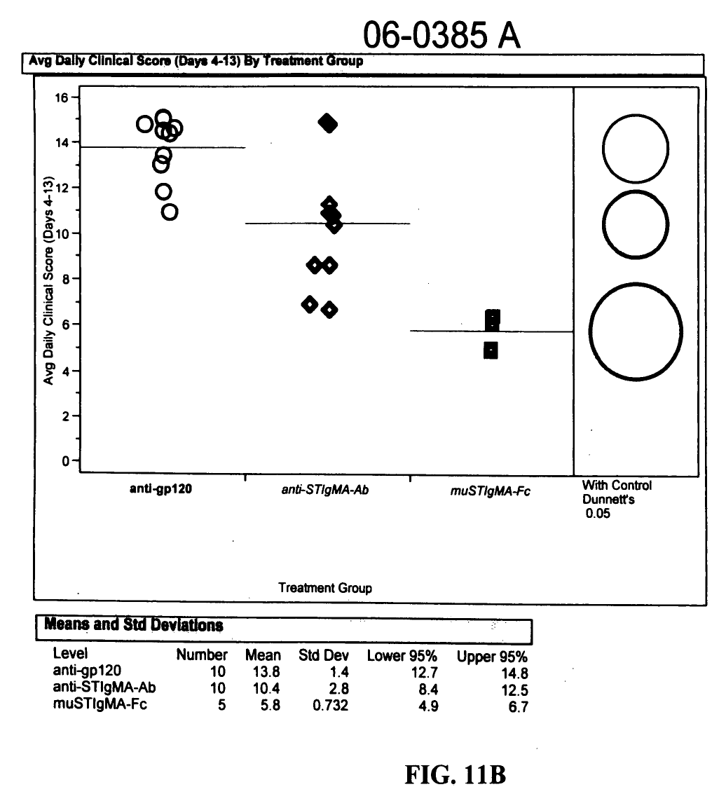

Treatment with a blocking mouse CRIg antibody inhibits paw swelling in a mouse model of passive arthritis (FIG. 11A, B.). All animals were held under Sterile Pathogen Free conditions and animal experiments were approved by the Institutional Animal Care and Use Committee of Genentech. Arthritis was induced by injection with an arthritogenic monoclonal antibody mixture (Chemicon) in six-week-old Balb/c mice (The Jackson Laboratory). Briefly, 2 mg of anti collagen type 11 antibodies was injected intraveneously in mice followed 3 days later by intraperitoneal injection with 25 μg of LPS. Mice were treated with 4 mg/kg CRIg-Fc or an antibody to gp-120 (IgG1 isotype, control-Fc) starting the day prior to antibody injection. Clinical scoring was performed by trained personnel blinded to the nature of the treatment

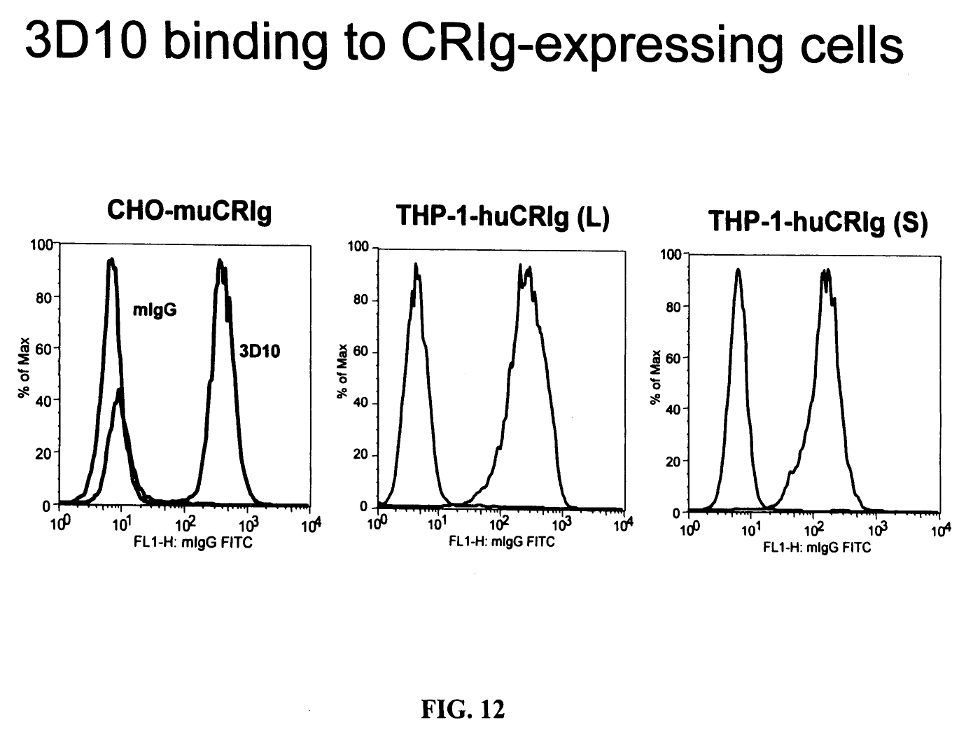

A blocking antibody that binds to both mouse and human CRIg. CHO cells expressing murine CRIg (muCRIg) or the short and long version of human CRIg (huCRIg (S), huCRIg (L)) were incubated with Alexa-488-conjugated antibody for 25 min at 4° C. and, washed and analyzed on FACScan. The results are shown in FIG. 12.

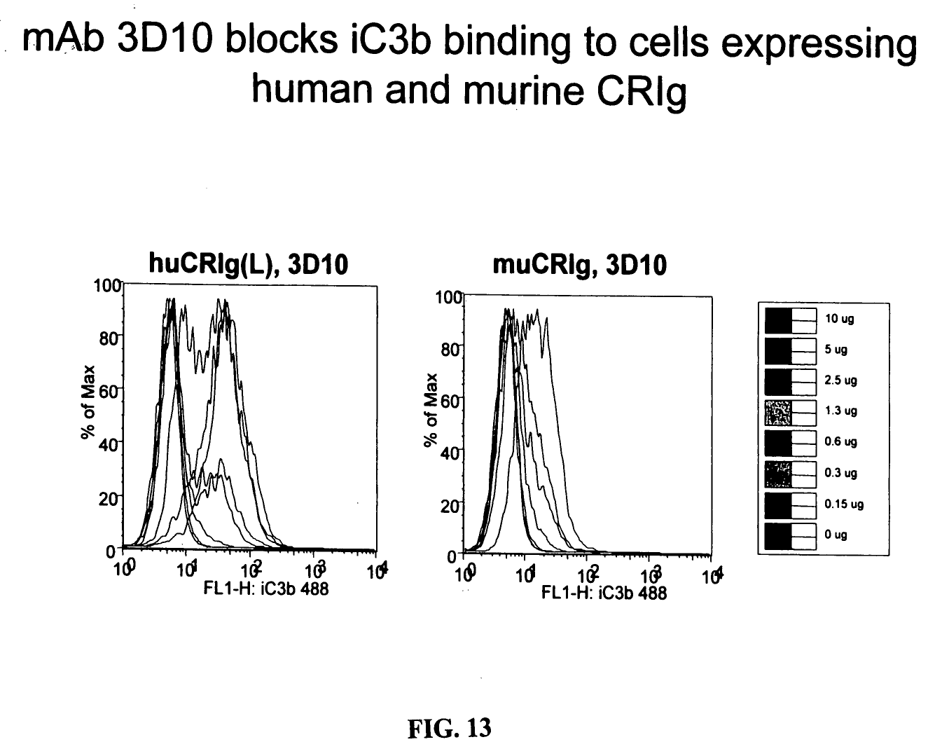

Inhibition of mouse and human CRIg binding to C3 fragments with a cross-blocking monoclonal antibody. Inhibition of iC3b binding to murine CRIg-expressing CHO cells and human CRIg-expressing THP-1 cells incubated with anti CRIg blocking antibodies: 400,000 CHO cells or THP-1 were coated with the antibodies for 25 min at 4° C. and washed 1×. 5 μg/ml (27 nM) iC3b was added for 30 min at 4° C., washed and analyzed on FACScan. The results are shown in FIG. 13.

Table 1 summarizes the characterization of CRIg antibodies based on binding to CRIg fusion protein coated on a maxisorp plate, binding to CRIg on the cell surface monitored by flow cytometry, and blocking of CRIg-C3b/iC3b interaction.

cDNAs encoding anti-CRIg antibodies were then deposited under the terms of the Budapest Treaty with the American Type Culture Collection, 10801 University Blvd., Manassas, Va. 20110-2209, USA (ATCC).

| Material | ATCC Deposit No. | Deposit date |
|---|---|---|
| 14G8 | PTA-8298 | Mar. 27, 2007 |
| 3D10 | PTA-8299 | Mar. 27, 2007 |
| 2H1 | PTA-8300 | Mar. 27, 2007. |

These deposits were made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposits will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC § 122 and the Commissioner's rules pursuant thereto (including 37 CFR § 1.14 with particular reference to 8860G 638). The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

All patent and literature references cited in the present specification are hereby expressly incorporated by reference in their entirety.

While the present invention has been described with reference to what are considered to be the specific embodiments, it is to be understood that the invention is not limited to such embodiments. To the contrary, the invention is intended to cover various modifications and equivalents included within the spirit and scope of the appended claims.

TABLE 1
Summary of antibody characteristics

| purified | | FACS | FACS | FACS | Block iC3b | Block iC3b | Block iC3b |
|---|---|---|---|---|---|---|---|
| clone | isotype | mCRIg CHO | hCRIg(S) THP-1 | hCRIg(L) THP-1 | mCRIg CHO | hCRIg(S) THP-1 | hCRIg(L) THP-1 |
| 3D10 | mIgG1 | ++ | ++ | ++ | + | + | + |
| 2H1 | mIgG1 | ++ | − | − | + | − | − |
| 14G8 | mIgG1 | ++ | − | − | + | − | − |

What is claimed is:

1. A CRIg antagonist blocking the binding of a native sequence CRIg polypeptide to C3b and/or iC3b, and inhibiting CRIg-mediated complement-dependent cellular entry of an intracellular pathogen or clearing an intracellular pathogen from the circulation.

2. The CRIg antagonist of claim 1 which is an anti-CRIg antibody or a fragment thereof.

3. The CRIg antagonist of claim 2 which is a monoclonal antibody or a fragment thereof.

4. The CRIg antagonist of claim 3 wherein the antibody fragment is selected from the group consisting of Fab, Fab′, F(ab′)₂, scFv, (scFv)₂, dAb, complementarity determining region (CDR) fragments, linear antibodies, single-chain antibody molecules, minibodies, diabodies, and multispecific antibodies formed from antibody fragments.

5. The CRIg antagonist of claim 3 which is a monoclonal antibody binding essentially to the same epitope as an anti-CRIg antibody selected from the group consisting of 14G8 (ATCC Deposit No. PTA-8298), 3D10 (ATCC Deposit No. PTA-8299) and 2H1 (ATCC Deposit No. PTA-8300), or a fragment thereof.

6. The CRIg antagonist of claim 3 which is a monoclonal anti-CRIg antibody selected from the group consisting of 14G8 (ATCC Deposit No. PTA-8298), 3D10 (ATCC Deposit No. PTA-8299) and 2H1 (ATCC Deposit No. PTA-8300), or a fragment thereof.

7. The CRIg antagonist of claim 3 wherein said antibody is chimeric, humanized or human, or a fragment thereof.

8. The CRIg antagonist of claim 7 wherein said antibody is a humanized antibody, or a fragment thereof.

9. The CRIg antagonist of claim 1 wherein said intracellular pathogen is selected from the group consisting of viruses, parasites, bacteria, fungi and prions.

10. The CRIg antagonist of claim 9 wherein said pathogen is an RNA or DNA virus.

11. The CRIg antagonist of claim 10 wherein said virus is selected from the group consisting of human immunodeficiency virus (HIV), hepatitis A, B, and C virus, herpes simplex virus (HSV), cytomegalovirus (CMV), Epstein-Barr virus (EBV), and human papilloma virus (HPV)).

12. The CRIg antagonist of claim 9 wherein said pathogen is a parasite.

13. The CRIg antagonist of claim 9 wherein said pathogen is a bacterium.

14. The CRIg antagonist of claim 9 wherein said pathogen is a fungus.

15. The CRIg antagonist of claim 9 wherein said pathogen is a prion.

16. Nucleic acid encoding an antibody of claim 3, or a fragment thereof.

17. A composition comprising a CRIg antagonist of claim 1 or claim 3 in admixture with a carrier.

18. The composition of claim 16 which is a pharmaceutical composition.

19. A method for inhibiting CRIg-mediated complement-dependent cellular entry of an intracellular pathogen, comprising administering to a mammalian subject in need an effective amount of a CRIg antagonist.

20. The method of claim 19 wherein said mammalian subject is a human.

21. The method of claim 20 wherein said CRIg antagonist is an anti-CRIg antibody or a fragment thereof.

22. A method for clearing an intracellular pathogen from the circulation of a mammalian subject, comprising administering to said subject an effective amount of a CRIg antagonist.

23. The method of claim 22 wherein said mammalian subject is a human.

24. The method 23 wherein said CRIg antagonist is an anti-CRIg antibody or a fragment thereof.

25. A method for the prevention or treatment of an infectious disease comprising administering to a mammalian subject in need an effective amount of a CRIg antagonist.

26. The method of claim 25 wherein said CRIg antagonist is an anti-CRIg antibody or a fragment thereof.

27. A kit comprising a CRIg antagonist and instructions for administering said CRIg antagonist to treat an infectious disease.


SEQUENCE LISTING (6 sequences)

SEQ ID NO: 1 - DNA, 1372 bp, Homo sapiens
  CDS (65)..(1261)
ccaactgcacctcggttctatcgataggaggctggaagaaaggacagaagtagctctggctgtgatggggatcttactgggcctgctactcctggggcacctaacagtggacacttatggccgtcccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcagactcctgatggcaaccaagtcgtgagagataagattactgagctccgtgtccagaaactctctgtctccaagcccacagtgacaactggcagcggttatggcttcacggtgccccagggaatgaggattagccttcaatgccaggctcggggttctcctcccatcagttatatttggtataagcaacagactaataaccaggaacccatcaaagtagcaaccctaagtaccttactcttcaagcctgcggtgatagccgactcaggctcctatttctgcactgccaagggccaggttggctctgagcagcacagcgacattgtgaagtttgtggtcaaagactcctcaaagctactcaagaccaagactgaggcacctacaaccatgacataccccttgaaagcaacatctacagtgaagcagtcctgggactggaccactgacatggatggctaccttggagagaccagtgctgggccaggaaagagcctgcctgtctttgccatcatcctcatcatctccttgtgctgtatggtggtttttaccatggcctatatcatgctctgtcggaagacatcccaacaagagcatgtctacgaagcagccagggcacatgccagagaggccaacgactctggagaaaccatgagggtggccatcttcgcaagtggctgctccagtgatgagccaacttcccagaatctgggcaacaactactctgatgagccctgcataggacaggagtaccagatcatcgcccagatcaatggcaactacgcccgcctgctggacacagttcctctggattatgagtttctggccactgagggcaaaagtgtctgttaaaaatgccccattaggccaggatctgctgacataatctagagtcgacctgcagaagcttggccgccatggcccaacttgtttattgcagcttataatggttacaaataa

SEQ ID NO: 2 - protein, 399 aa, Homo sapiens
MGILLGLLLLGHLTVDTYGRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLPVFAIILIISLCCMVVFTMAYIMLCRKTSQQEHVYEAARAHAREANDSGETMRVAIFASGCSSDEPTSQNLGNNYSDEPCIGQEYQIIAQINGNYARLLDTVPLDYEFLATEGKSVC

SEQ ID NO: 3 - DNA, 1090 bp, Homo sapiens
  CDS (67)..(981)
gtccaactgcacctcggttctatcgataggaggctggaagaaaggacagaagtagctctggctgtgatggggatcttactgggcctgctactcctggggcacctaacagtggacacttatggccgtcccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcagactcctgatggcaaccaagtcgtgagagataagattactgagctccgtgtccagaaacactcctcaaagctactcaagaccaagactgaggcacctacaaccatgacataccccttgaaagcaacatctacagtgaagcagtcctgggactggaccactgacatggatggctaccttggagagaccagtgctgggccaggaaagagcctgcctgtctttgccatcatcctcatcatctccttgtgctgtatggtggtttttaccatggcctatatcatgctctgtcggaagacatcccaacaagagcatgtctacgaagcagccagggcacatgccagagaggccaacgactctggagaaaccatgagggtggccatcttcgcaagtggctgctccagtgatgagccaacttcccagaatctgggcaacaactactctgatgagccctgcataggacaggagtaccagatcatcgcccagatcaatggcaactacgcccgcctgctggacacagttcctctggattatgagtttctggccactgagggcaaaagtgtctgttaaaaatgccccattaggccaggatctgctgacataatctagagtcgacctgcagaagcttggccgccatggcccaacttgtttattgcagcttataatggttacaata

SEQ ID NO: 4 - protein, 305 aa, Homo sapiens
MGILLGLLLLGHLTVDTYGRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKHSSKLLKTKTEAPTTMTYPLKATSTVKQSWDWTTDMDGYLGETSAGPGKSLPVFAIILIISLCCMVVFTMAYIMLCRKTSQQEHVYEAARAHAREANDSGETMRVAIFASGCSSDEPTSQNLGNNYSDEPCIGQEYQIIAQINGNYARLLDTVPLDYEFLATEGKSVC

SEQ ID NO: 5 - DNA, 1590 bp, Mus musculus
  CDS (134)..(973)
gtccaactgcacctcggttctatcgattcgaattcggccacactggccggatcctctagagatccctcgacctcgacccacgcgtccgagcagcaagaggatggaaggatgaatagaagtagcttcaaataggatggagatctcatcaggcttgctgttcctgggccacctaatagtgctcacctatggccaccccaccctaaaaacacctgagagtgtgacagggacctggaaaggagatgtgaagattcagtgcatctatgatcccctgagaggctacaggcaagttttggtgaaatggctggtaagacacggctctgactccgtcaccatcttcctacgtgactccactggagaccatatccagcaggcaaagtacagaggccgcctgaaagtgagccacaaagttccaggagatgtgtccctccaaataaataccctgcagatggatgacaggaatcactatacatgtgaggtcacctggcagactcctgatggaaaccaagtaataagagataagatcattgagctccgtgttcggaaatataatccacctagaatcaatactgaagcacctacaaccctgcactcctctttggaagcaacaactataatgagttcaacctctgacttgaccactaatgggactggaaaacttgaggagaccattgctggttcagggaggaacctgccaatctttgccataatcttcatcatctccctttgctgcatagtagctgtcaccataccttatatcttgttccgctgcaggacattccaacaagagtatgtctatggagtgagcagggtgtttgccaggaagacaagcaactctgaagaaaccacaagggtgactaccatcgcaactgatgaaccagattcccaggctctgattagtgactactctgatgatccttgcctcagccaggagtaccaaataaccatcagatcaacaatgtctattcctgcctgctgaacacagtttccagaaactaagaagttcttgctactgaagaaaataacatctgctaaaatgcccctactaagtcaaggtctactggcgtaattacctgttacttatttactacttgccttcaacatagctttctccctggcttcctttcttcttagacaacctaaagtatctatctagtctgccaattctggggccattgagaaatcctgggtttggctaagaatatactacatgcacctcaagaaatctagcttctgggcttcacccagaacaattttcttcctagggccttcacaactcttctccaaacagcagagaaattccatagcagtagaggttctttatcatgcctccagacagcgtgagtctcagtcctacaaactcagacaagcacatgggtctaggattactcctctttctctagggccagatgacttttaattgatattactattgctacattatgaatctaatgcacatgtattcttttgttgttaataaatgtttaatcatgacatcaaaaaaaaaaaaaaaaagggcggccgcgactctagagtcgacctgcagtagggataacagggtaataagcttggccgccatggcccaacttgttt

SEQ ID NO: 6 - protein, 280 aa, Mus musculus
MEISSGLLFLGHLIVLTYGHPTLKTPESVTGTWKGDVKIQCIYDPLRGYRQVLVKWLVRHGSDSVTIFLRDSTGDHIQQAKYRGRLKVSHKVPGDVSLQINTLQMDDRNHYTCEVTWQTPDGNQVIRDKIIELRVRKYNPPRINTEAPTTLHSSLEATTIMSSTSDLTTNGTGKLEETIAGSGRNLPIFAIIFIISLCCIVAVTIPYILFRCRTFQQEYVYGVSRVFARKTSNSEETTRVTTIATDEPDSQALISDYSDDPCLSQEYQITIRSTMSIPAC